(19) United States
(12) Patent Application Publication
(10) Pub. No.: US 2006/0030540 A1
(43) Pub. Date: Feb. 9, 2006

(54) Transgenic animals produced using oral administration of a genetic agent coupled to a transporting agent

(75) Inventors: Gonzalo Hortelano, Burlington (CA); Andrew Gomez Vargas, Dundas (CA)

Correspondence Address: MCHALE & SLAVIN, P.A., 2855 PGA BLVD, PALM BEACH GARDENS, FL, 33410, US

(73) Assignee: Neox, Inc., Burlington (CA)

(21) Appl. No.: 11/244,782

(22) Filed: Oct. 6, 2005

Related U.S. Application Data
(63) Continuation of application No. 10/323,348, filed on Dec. 18, 2002.
Application No. 10/323,348 is a continuation-in-part of application No. 10/199,914, filed on Jul. 18, 2002.

(51) Int. Cl. A61K 48/00; A01K 67/027

(52) U.S. Cl. 514/44; 800/21

(57) ABSTRACT

The present invention is directed toward a method of producing transgenic animals by administration of a composition to an animal via a natural gastrointestinal pathway. The composition achieves widespread distribution, systemic expression and sustained delivery in the animal. More particularly, the invention discloses a method for producing a transgenic animal using oral gene therapy. The present invention also provides transgenic animals produced by this method.

### CROSS REFERENCE TO RELATED APPLICATION

This application is a continuation of application Ser. No. 10/323,348, filed Dec. 18, 2002, which is a continuation-in-part of application Ser. No. 10/199,914 filed on Jul. 18, 2002, currently pending and incorporated herein by reference.

### FIELD OF THE INVENTION

The instant invention relates to the production of transgenic animals by the oral administration of a composition to an animal; particularly to the production of transgenic animals by the administration of a genetic agent coupled to a transporting agent; and most particularly to the production of transgenic animals by the widespread distribution, systemic expression and sustained delivery of a genetic agent via oral administration when effectively coupled to a polypeptide carrier.

### BACKGROUND OF THE INVENTION

A conventional transgenic animal is defined as an animal having an exogenous gene (transgene) introduced into the germline of the animal or an ancestor thereof, typically in the single cell stage of development. A transgene is a nucleotide sequence that is artificially integrated into the genome of a cell and is used to transform the cell for the purpose of achieving a genetic change distinct from the “normal” or “wild-type” state. A transgenic animal is then developed from these transformed cells. Various types of nucleotide sequences can be used to generate transgenic animals, such as mutant sequences or heterologous (exogenous) sequences. Transgenic animal models are useful as tools to study protein function, especially in disease states. Several methods to produce transgenic animals have become conventional in the art; one such method is production by homologous recombination wherein all or part of the genome is replaced by homologous sequences. “Knock-out” animals can also be produced, for example, using homologous recombination wherein an entire gene is eliminated from the genome in order to assess function. Alternatively, “knock-in” animals can be produced wherein a gene function is introduced into a genome. “Knock-in” and “knock-out” techniques may be combined to generate one transgenic animal; see U.S. Pat. No. 6,060,642. Transgenic animals described as “null mutants” can also be generated by mutating a protein so that it no longer functions as the native protein; see U.S. Pat. No. 5,557,032. Microinjection of embryos with genetic material is another method known to be useful for the generation of transgenic animals; see U.S. Pat. No. 4,736,866. A transgenic animal produced by any of the above described conventional methods will incorporate the exogenous gene into all of its cells (both germline and somatic) and will pass the exogenous gene on to its progeny.

There are several disadvantages to conventional methods for the production of transgenic animals; a) generation of the transgenic animal is expensive and involves complex and time-consuming experimentation, for example, the time to prepare a construct, length of gestation periods before offspring can be obtained and the need for multiple generations to achieve the desired animal (heterozygous chimeras must be bred to obtain homozygous individuals) are all issues for consideration and b) since the transgenic animal can pass the transgene on to its progeny the danger exists that the transgene can contaminate the “wild-type” population through uncontrolled breeding with possible deleterious effects on the integrity of both experimental results and the natural population. The instant inventors have discovered that their method for oral delivery of DNA leads to long-term expression of human (exogenous) transgenes in animals, accordingly, the instant invention utilizes this method for production of an animal wherein exogenous or foreign DNA has been incorporated to essentially all cells, whereby an animal which is, in essentially all respect, equivalent to a transgenic animal is produced, while avoiding the disadvantages described above. Animals produced in accordance with this invention will heretofore be referred to as transgenic animals.

Gene therapy offers an alternative to the currently available treatment modalities for a variety of conditions, particularly genetic and acquired disorders affecting a range of cells and tissues. There exist ex vivo approaches based upon the implantation of autologous genetically-modified cells. Several in vivo gene therapy protocols based on viral vectors are known, albeit several safety related issues exist, for example, immunologic responses and the introduction of potential pathologic viral sequences. Oral gene delivery has been attempted with little success, largely due to the extensive degradation of DNA in the gastrointestinal tract. Attempts at oral gene therapy via the use of liposomal formulations as a protectant has met with limited success, in that the efficiency of delivery is relatively low.

Additionally as in the production of transgenic animals, germ line transmission of DNA is a concern in gene therapy protocols (E. Marshall Science 294:1640 2001 and E. Marshall Science 294:2268 2001). There are the potential dangers of passing on the new trait to future human generations or fortuitously spreading a particular transgene into the environment.

Although various methods have been attempted, with an eye toward distribution of DNA via oral administration, what has eluded prior artisans is a process and a device which enables widespread distribution of DNA throughout all organs and tissues via oral administration without eliciting an immune response, whereby persistent and efficient protein expression is accomplished.

### DESCRIPTION OF THE PRIOR ART

Quong et al., in an article entitled “DNA Protection from Extracapsular Nucleases, within Chitosan or Poly-L-lysine-coated Alginate Beads” (Biotechnology and Bioengineering, Vol. 60, No. 1, October 1998, pages 124-134, 1998) discloses immobilization of DNA within an alginate matrix using either an internal or external source of calcium followed by membrane coating with chitosan or poly-L-lysine (PLL). The work carried out by Quong et al. concluded that PLL coating provides enhanced protection of DNA against DNase in vitro when compared to uncoated beads.

Ward et al. (Blood, 15 Apr. 2001, Volume 97, Number 8, Pages 2221-2229) is directed toward intravenous forms of gene therapy capable of systemic circulation. Complexes of poly L-lysine (PLL) have been targeted to various cell lines in vitro by covalent attachment of targeting ligands to the PLL, resulting in transgene expression. Ward characterizes these complexes as having little use in vivo since they have poor circulatory half-lives. Ward further theorizes that since complexes activate human complement in vitro and stimulate the immune system, this most likely accounts for their poor half-life in vivo. Thus, this work fails to disclose any form of widespread transgene distribution or expression (of proteins, antibodies or the like coded products) via this methodology.

Rothbard et al. (Nature Medicine, Volume 6, Number 11, November 2000, Pp. 1253-1257) discloses the conjugation of arginine and cyclosporin-A to form a compound useful in traversing the stratum corneum and thereby entering the epidermis. The disclosed process is useful in forming a conjugate which, unlike cyclosporin-A alone, is capable of reaching dermal T lymphocytes and inhibiting cutaneous inflammation. The reference fails to teach or suggest the conjugation of DNA to arginine, nor does it in any way contemplate oral ingestion of a conjugated arginine of any kind.

Wender et al. (PNAS USA, Nov. 21, 2000, vol. 97, no. 24, 13003-13008) discloses polyguanidine peptoid derivatives which preserve the 1,4-backbone spacing of side chains of arginine oligomers to be efficient molecular transporters as evidenced by cellular uptake. While it is suggested that these peptoids could serve as effective transporters for the molecular delivery of drugs, drug candidates, and agents into cells, the reference is nevertheless silent as to the concept of oral delivery via this route, and does not disclose the formation of a complex between the active ingredient, e.g. DNA or a drug, and the polyguanidine peptoid derivatives.

One of the instant inventors is co-author of a series of articles related to gene therapy. In an article in Human Gene Therapy, (6:165-175 (February 1995) Al-Hendy et al.) nonautologous somatic gene therapy via the use of encapsulated myoblasts secreting mouse growth hormone to growth hormone deficient Snell dwarf mice is disclosed. Immunoprotective alginate-poly-l-lysine-alginate microcapsules were used to protect recombinant allogeneic cells from rejection subsequent to their implantation. Oral gene therapy is neither contemplated nor suggested.

In Blood, Vol. 87, No. 12, Jun. 15, 1996, Pp. 5095-5103, Hortelano et al. disclose delivery of Human Factor IX by use of encapsulated recombinant myoblasts. Droplets of an alginate-cell mixture were collected in a calcium chloride solution. Upon contact, the droplets gelled. Subsequently, the outer alginate layer was cross-linked with poly-L-lysine hydrobromide (PLL) and then with another layer of alginate. The remaining free alginate core was then dissolved via sodium citrate to yield microcapsules with an alginate-PLL-alginate membrane containing cells. Similar technology is disclosed in Awrey et al., Biotechnology and Bioengineering, Vol. 52, Pp. 472-484 (1996), Peirone et al., Encapsulation of Various Recombinant Mammalian Cell Types in Different Alginate microcapsules, Journal of Biomedical Materials Research 42(4):587-596, 1998), and in Haemophilia (2001), 7, 207-214. The references neither disclose nor suggest the use of immuno-isolation devices for the delivery of gene therapy via an oral route.

In an article by Chang et al., Tibtech/Trends in Biotechnology, 17(2); February 1999, entitled “The in Vivo Delivery of Heterologous Proteins by Microencapsulated Recombinant Cells” the use of microencapsulated E-Coli engineered to express Klebsiella aerogens urease gene was administered orally. It is disclosed that passage of the live bacteria via the gastrointestinal tract was found to permit the clearance of urea, thereby lowering the plasma urea levels. This disclosure is not suggestive of the use of oral gene therapy to result in widespread dissemination of DNA via an oral pathway.

Brown et al., “Preliminary Characterization of Novel Amino Acid Based Polymeric Vesicles as Gene and Drug Delivery Agents” (Bioconjugate Chem. 2000, 11, 880-891) teaches formation of an amphiphilic polymer matrix using poly-L-lysine with polyethylene glycol modification, as a means of gene delivery to a cell in vivo. The disclosure is directed toward transfer of DNA into live cells when incorporated within PLL-PEG vesicles. The disclosure fails to teach oral administration, nor the combination of an GI tract protector, such as alginate, in combination with a polypeptide suitable for use as a DNA transporting agent in accordance with the teachings of the instant invention.

Leong et al., “Oral Gene Delivery With Chitosan-DNA Nanoparticles Generates Immunologic Protection In A Murine Model Of Peanut Allergy” (Nature Medicine, Volume 5, Number 4, April 1999, Pp 387-391) discloses chitosan/DNA nanoparticles synthesized by complexing plasmid DNA with chitosan for oral ingestion to treat allergic response to peanut antigen. The reference fails to show widespread distribution, in that staining only showed gene expression in the stomach and small intestine.

U.S. Pat. No. 6,217,859 discloses a composition for oral administration to a patient for removal of undesirable chemicals or amino acids caused by disease. The composition comprises entrapped or encapsulated microorganisms capable of removing the undesired chemicals or amino acids. The capsules may comprise a variety of polymers, elastomers, and the like, inclusive of which are chitosan-alginate and alginate-polysine-alginate compounds.

U.S. Pat. No. 6,177,274 is directed toward a compound for targeted gene delivery consisting of polyethylene glycol (PEG) grafted poly (L-lysine) and a targeting moiety. The polymeric gene carriers of this invention are capable of forming stable and soluble complexes with nucleic acids, which are in turn able to efficiently transform cells. The reference fails to suggest or disclose a complex including DNA, nor the use of such a complex for oral delivery thereof.

U.S. Pat. No. 6,258,789 is directed towards a method of delivering a secreted protein into the bloodstream of a mammalian subject. In the disclosed method, intestinal epithelial cells of a mammalian subject are genetically altered to operatively incorporate a gene which expresses a protein which has a desired effect. The method of the invention comprises administration of a formulation containing DNA to the gastrointestinal tract, preferably by an oral route. The expressed recombinant protein is secreted directly into the bloodstream. Of particular interest is the use of the method of the invention to provide for short term, e.g. two to three days, delivery of gene products to the bloodstream.

U.S. Pat. No. 6,255,289 discloses a method for the genetic alteration of secretory gland cells, particularly pancreatic and salivary gland cells, to operatively incorporate a gene which expresses a protein which has a desired therapeutic effect on a mammalian subject. The expressed protein is secreted directly into the gastrointestinal tract and/or blood stream to obtain therapeutic blood levels of the protein thereby treating the patient in need of the protein. The transformed secretory gland cells provide long term therapeutic cures for diseases associated with a deficiency in a particular protein or which are amenable to treatment by over-expression of a protein.

U.S. Pat. No. 6,225,290 discloses a process wherein the intestinal epithelial cells of a mammalian subject are genetically altered to operatively incorporate a gene which expresses a protein which has a desired therapeutic effect. Intestinal cell transformation is accomplished by administration of a formulation composed primarily of naked DNA. Oral or other intragastrointestinal routes of administration provide a method of administration, while the use of naked nucleic acid avoids the complications associated with use of viral vectors to accomplish gene therapy. The expressed protein is secreted directly into the gastrointestinal tract and/or bloodstream to obtain therapeutic blood levels of the protein thereby treating the patient in need of the protein. The transformed intestinal epithelial cells provide short or possibly long term therapeutic cures (e.g. short term being up to about 2-4 days, while long-term, via incorporation in intestinal villi is theorized to possibly last for weeks or months) for diseases associated with a deficiency in a particular protein or which are amenable to treatment by overexpression of a protein. It is noted, however, that the expression is limited to within the gastrointestinal tract, thus relegating distribution of the expressed entity to the bloodstream, where immunogenic response and resulting neutralization of said entity via the immune system becomes problematic.

U.S. Pat. No. 5,837,693 is directed to intravenous hormone polypeptide delivery by salivary gland expression. Secretory gland cells, particularly pancreatic and salivary gland cells, are genetically altered to operatively incorporate a gene which expresses a protein which has a desired therapeutic effect on a mammalian subject. The expressed protein may be secreted directly into the gastrointestinal tract and/or blood stream. The transformed secretory gland cells may provide therapeutic cures for diseases associated with a deficiency in a particular protein or which are amenable to treatment by over-expression of a protein.

U.S. Pat. No. 5,885,971 is directed toward gene therapy by secretory gland expression. Secretory gland cells, particularly pancreatic and salivary gland cells, are genetically altered to operatively incorporate a gene which expresses a protein which has a desired therapeutic effect on a mammalian subject. The expressed protein may be secreted directly into the gastrointestinal tract and/or blood stream to obtain therapeutic blood levels of the protein thereby treating the patient in need of the protein. The transformed secretory gland cells provide long term therapeutic cures for diseases associated with a deficiency in a particular protein or which are amenable to treatment by over-expression of a protein.

U.S. Pat. No. 6,004,944 is directed to protein delivery via secretory gland expression. Secretory gland cells, particularly pancreatic, hepatic, and salivary gland cells, are genetically altered to operatively incorporate a gene which expresses a protein which has a desired therapeutic effect on a mammalian subject. The expressed protein may be secreted directly into the bloodstream to obtain therapeutic levels of the protein thereby treating the patient in need of the protein. The transformed secretory gland cells may provide long term or short term therapies for diseases associated with a deficiency in a particular protein or which are amenable to treatment by over-expression of a protein.

U.S. Pat. No. 6,008,336 relates to compacted nucleic acids and their delivery to cells. Nucleic acids are compacted, substantially without aggregation, to facilitate their uptake by target cells of an organism to which the compacted material is administered. The nucleic acids may achieve a clinical effect as a result of gene expression, hybridization to endogenous nucleic acids whose expression is undesired, or site-specific integration so that a target gene is replaced, modified or deleted. The targeting may be enhanced by means of a target cell-binding moiety. The nucleic acid is preferably compacted to a condensed state. In one embodiment, nucleic acid complexes are consisting essentially of a single double-stranded cDNA molecule and one or more polylysine molecules, wherein said cDNA molecule encodes at least one functional protein, wherein said complex is compacted to a diameter which is less than double the theoretical minimum diameter of a complex of said single cDNA molecule and a sufficient number of polylysine molecules to provide a charge ratio of 1:1, in the form of a condensed sphere, wherein the nucleic acid complexes are associated with a lipid.

U.S. Pat. No. 6,287,817 discloses a protein conjugate consisting of antibody directed at the pIgR and A₁ AT which can be transported specifically from the basolateral surface of epithelial cells to the apical surface. This approach provides the ability to deliver a therapeutic protein directly to the apical surface of the epithelium, by targeting the pIgR with an appropriate ligand.

U.S. Pat. No. 6,261,787 sets forth a bifunctional molecule consisting of a therapeutic molecule and a ligand which specifically binds a transcytotic receptor; said bifunctional molecule can be transported specifically from the basolateral surface of epithelial cells to the apical surface. This approach provides the ability to deliver a therapeutic molecule directly to the apical surface of the epithelium, by targeting the transcytotic receptor with an appropriate ligand.

U.S. Pat. No. 5,877,302 is directed toward compacted nucleic acids and their delivery to cells. Nucleic acids are compacted, substantially without aggregation, to facilitate their uptake by target cells of an organism to which the compacted material is administered. The nucleic acids may achieve a clinical effect as a result of gene expression, hybridization to endogenous nucleic acids whose expression is undesired, or site-specific integration so that a target gene is replaced, modified or deleted. The targeting may be enhanced by means of a target cell-binding moiety, e.g. polylysine. The nucleic acid is preferably compacted to a condensed state.

U.S. Pat. No. 6,159,502 relates to an oral delivery system for microparticles. There are disclosed complexes and compositions for oral delivery of a substance or substances to the circulation or lymphatic drainage system of a host. The complexes of the invention comprise a microparticle coupled to at least one carrier, the carrier being capable of enabling the complex to be transported to the circulation or lymphatic drainage system via the mucosal epithelium of the host, and the microparticle entrapping or encapsulating, or being capable of entrapping or encapsulating, the substance(s). Examples of suitable carriers are mucosal binding proteins, bacterial adhesins, viral adhesins, toxin binding subunits, lectins, Vitamin B₁₂ and analogues or derivatives of Vitamin B₁₂ possessing binding activity to Castle's intrinsic factor. This invention differs from the instant disclosure in requiring entrapment or encapsulation, which neither insures nor enables the widespread distribution, systemic expression, or sustained delivery which are novel features of the instantly disclosed invention.

U.S. Pat. No. 6,011,018 discloses regulated transcription of targeted genes and other biological events. Dimerization and oligomerization of proteins are general biological control mechanisms that contribute to the activation of cell membrane receptors, transcription factors, vesicle fusion proteins, and other classes of intra- and extracellular proteins. The patentees have developed a general procedure for the regulated (inducible) dimerization or oligomerization of intracellular proteins. In principle, any two target proteins can be induced to associate by treating the cells or organisms that harbor them with cell permeable, synthetic ligands. Regulated intracellular protein association with these cell permeable synthetic ligands are deemed to offer new capabilities in biological research and medicine, in particular, in gene therapy. Using gene transfer techniques to introduce these artificial receptors, it is indicated that one may turn on or off the signaling pathways that lead to the over-expression of therapeutic proteins by administering orally active “dimerizers” or “de-dimerizers”, respectively. Since cells from different recipients can be configured to have the pathway over-express different therapeutic proteins for use in a variety of disorders, the dimerizers have the potential to serve as “universal drugs”. They can also be viewed as cell permeable, organic replacements for therapeutic antisense agents or for proteins that would otherwise require intravenous injection or intracellular expression (e.g., the LDL receptor or the CFTR protein).

What is lacking in the art is an orally deliverable composition capable of achieving: a) widespread delivery and distribution of a therapeutic agent such as DNA, to essentially all cells of the targeted subject; b) an ability to provide a sustained (e.g. non-transient) expression of a therapeutic moiety by said therapeutic agent (either ubiquitously or in a tissue specific manner), from a single administration, via cellular uptake in virtually all organs and cellular systems throughout the entire body, and c) without eliciting an unwanted immune response. The instant inventors have developed a composition and a methodology to accomplish a, b and c as noted above in the instant paragraph; said composition and methodology are claimed in the parent application Ser. No. 10/199,914, filed on Jul. 18, 2002 which is incorporated herein by reference. While working on these concepts, the instant inventors realized that transgenic animals can be produced using this composition and methodology which circumvent some of the disadvantages of conventional methods for the production of transgenic animals noted above.

### SUMMARY OF THE INVENTION

The present invention is directed toward both a novel method for producing animal models that are virtual transgenics and to animals produced by this method. More particularly, the present invention is directed toward both a method of producing transgenic animals by administration of a composition to an animal via a natural gastrointestinal pathway (oral gene therapy) and to animals produced by this method.

Various obstacles have prevented an efficient oral gene therapy protocol. The primary obstacle has been the extensive degradation of ingested DNA. Protecting this otherwise naked DNA from destruction when placed in the gastrointestinal tract, for example via the use of chitosan, collagen, alginate or the like, enables limited absorption of DNA via the gastrointestinal tract, albeit with limited scope of delivery and poor expression.

In order to achieve maximum distribution and efficacy via oral administration, it has been determined that DNA requires a protective covering. For example, alginate is a means of providing protection in the gastrointestinal tract. Additionally, a transporting agent is required, which is capable of transporting the DNA via natural pathways, and without eliciting an unwanted or undesirable immunogenic response during transport. The transporting agent, in its broadest sense, may be any compound containing an amine group that is capable of coupling with the DNA (or other therapeutic agent) in a manner effective to produce efficacious and widespread distribution and cellular uptake subsequent to passage via said natural gastrointestinal pathway. Such coupling of the therapeutic agent and transporting agent thereby enables efficacious and widespread absorption, distribution and expression thereof. In a particularly preferred embodiment, the transporting agent is preferably a polypeptide or a modification thereof, e.g. of an amino acid, but may be any compound having an amine group and an acidic group which will effectively enable in vivo distribution. The transporting agent is necessary in order to achieve efficient and widespread distribution of the therapeutic product, e.g. DNA in vivo. Thus, in a preferred embodiment, the instantly disclosed formulations will couple DNA to the amino compound, e.g. via electrostatic binding, while protecting the DNA from degradation in the gastrointestinal tract, e.g. with an alginate or equivalent protective compound. Such a formulation may be illustratively exemplified as an alginate cross-linked with poly-L-lysine, such as in the form of a nanoparticle. While the instant inventors have shown that limited expression is possible by merely protecting DNA in the GI tract via the use of gelatin or alginate, without PLL, or even via the administration of naked DNA, the effectivity is clearly much lower, and therefore inclusion of a protective agent and a transporting agent (e.g. alginate/PLL) is most preferred.

In order to make DNA microcapsules, DNA is first mixed with alginate or a compound having similar properties in affording GI tract protection for the DNA, then the capsules are physically formed with DNA-alginate inside, and later the transporting agent, e.g. PLL, is added to cross-link the alginate beads, in a manner such that conjugation or coupling between the transporting agent and DNA occurs, although the transport agent does not specifically encapsulate the therapeutic agent. Absent the presence of the transporting agent, e.g. PLL, the experiments indicate that there is no widespread distribution or delivery nor is there systemic or sustained expression. This evidences the theory that an interaction or coupling of the transporting agent and therapeutic agent occurs within the capsules, thereby explaining the efficacy of the instantly disclosed microcapsules in the distribution of DNA to all major organs.

Tissue-specific expression of therapeutic genes can be achieved by using tissue-specific genetic regulatory elements (promoters) that restrict gene expression to specific organs. Via the judicious use of promoters, the degree of expression may be tailored to meet specific needs. For example, via the use of β Actin, a ubiquitous promoter, widespread expression is achieved. Alternatively, use of tissue specific regulatory elements (promoters), for example, but not limited to albumin promoter (liver expression), muscle creatine kinase (MCK) for muscle expression, and keratinocyte (skin expression) provide the ability to express protein in a particularly desired portion of the body.

The instant inventors have shown that their method for oral delivery of DNA leads to long-term expression of transgenes in animals, thus they have achieved a method for production of transgenic animals using oral gene therapy eliminating the time normally required for gestation and birth of conventional transgenic animals. Additionally, the orally delivered transgene is not expressed in the germ cells of the transgenic animals thus preventing the danger of transgene contamination of the “wild-type” population through uncontrolled breeding with possible deleterious effects on the integrity of both experimental results and the natural population.

Accordingly, it is an objective of the instant invention to provide a process for production of a transgenic animal by systemic delivery of a complete transcriptional unit (for example, DNA and RNA, or components which enable a complete transcriptional unit within the cells, e.g. FIX cDNA coupled to a suitable promoter and polyadenylation signal) to virtually all cells of an organism, via an oral pathway.

It is a further objective of the instant invention to provide a process for production of a transgenic animal wherein the expression of the transgene in the transgenic animal is controllable (for example, ubiquitous or tissue specific) via said complete transcriptional unit in conjunction with judicious promoter selection.

It is still a further objective of the instant invention to provide a process for production of a transgenic animal by delivery of DNA and RNA to a variety of organs, including but not limited to heart, muscle, lungs, skin, kidney, liver, brain and spleen.

Additionally, it is an objective of the instant invention to provide transgenic animals made by any of the processes mentioned above as objectives of the instant invention and characterized by a lack of germ cell alteration, thereby precluding progenic transmission.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a fluorescent micrograph illustrating expression in the liver.

FIG. 2 is a fluorescent micrograph illustrating expression in the kidney.

FIG. 3 is a fluorescent micrograph illustrating expression in the lung.

FIG. 4 is a fluorescent micrograph illustrating expression in the heart.

FIG. 5 is a fluorescent micrograph illustrating expression in the muscle.

FIG. 6 is a fluorescent micrograph illustrating expression in the skin.

FIG. 7 is a fluorescent micrograph illustrating expression in the vessels.

FIG. 8 represents a graphical analysis of an in vitro assay of Activated Partial Thromboplastin Time (APTT).

FIG. 9 shows GFP DNA expression by PCR analysis in organs of mice fed GFP DNA and sacrificed on day 42 post ingestion.

FIG. 10 is a fluorescent micrograph illustrating expression utilizing arginine/ornithine transport agents in the duodenum.

FIG. 11 is a fluorescent micrograph illustrating expression utilizing arginine/ornithine transport agents in the jejunum.

FIG. 12 is a fluorescent micrograph illustrating expression utilizing arginine/ornithine transport agents in the Ileum.

FIG. 13 is a fluorescent micrograph illustrating expression utilizing arginine/ornithine transport agents in the colon.

FIG. 14 is a fluorescent micrograph illustrating expression utilizing arginine/ornithine transport agents in the liver.

FIG. 15 is a fluorescent micrograph illustrating expression utilizing arginine/ornithine transport agents in the spleen.

FIG. 16 is a fluorescent micrograph illustrating expression utilizing arginine/ornithine transport agents in the kidney.

FIG. 17 is a fluorescent micrograph illustrating expression utilizing arginine/ornithine transport agents in the lung.

FIG. 18 is a fluorescent micrograph illustrating expression utilizing arginine/ornithine transport agents in the heart.

FIG. 19 is a fluorescent micrograph illustrating expression utilizing arginine/ornithine transport agents in the muscle.

FIG. 20 is a fluorescent micrograph illustrating expression utilizing arginine/ornithine transport agents in the pancreas.

FIG. 21 is a fluorescent micrograph illustrating expression utilizing arginine/ornithine transport agents in the brain.

FIG. 22 is a fluorescent micrograph illustrating expression utilizing arginine/ornithine transport agents in the gonads.

FIG. 23 is a fluorescent micrograph illustrating expression utilizing arginine/ornithine transport agents in the skin.

FIG. 24 is a fluorescent micrograph illustrating expression utilizing arginine/ornithine transport agents in the vessels.

FIG. 25 is a fluorescent micrograph illustrating expression utilizing arginine/ornithine transport agents in the blood vessels.

FIG. 26 is a graphical representation showing the levels of hGH in treated mice.

FIG. 27 illustrates that anti-hGH antibodies were not detected post hGH production.

FIG. 28 is a fluorescent micrograph illustrating tissue specific expression in the liver utilizing an albumin promoter.

FIG. 29 is a bar graph comparing the level of hGH achieved using alternative technologies.

FIG. 30 is a graphical analysis over time of hGH levels achieved using alternative technologies.

FIG. 31 is illustrative of the presence of hGH in various organs achieved using alternative technologies.

FIG. 32 depicts weight gain attributable to hGH levels achieved using alternative technologies.

FIG. 33 is a schematic representation of the construct used for ubiquitous expression of Factor IX.

FIG. 34 is a schematic representation of the construct used for ubiquitous expression of GFP.

FIGS. 35A-I shows fluorescent micrographs illustrating GFP expression in various organs of mice fed GFP DNA and sacrificed on day 42 post-ingestion. A) Liver; B) Spleen; C) Lung; D) Heart; E) Muscle; F) Kidney; G) Skin; H) Brain and I) Vessel.

FIGS. 36A-B show amplification of DNA from murine organs using PCR. A) 42 days after treatment with oral GFP DNA and B) 400 days after treatment with oral hFIX DNA.

FIGS. 37A-B show biodistribution of hFIX expression in murine tissues. A) shows expression under the control of the ubiquitous β-actin promoter and B) expression under the control of the liver-specific albumin promoter.

FIG. 38 is a graphical representation of hGH expression in mice orally treated with hGH DNA. FIG. 39 is a graphical representation of sustained delivery of α-1 antitrypsin in a dose response manner in mice treated with oral DNA.

FIGS. 40A-C show the reversal of hemophilia A) shows graphical representation of the APTT time in treated mice; B) shows bleeding time following tail transection and C) shows bleeding time following tail transection of mice treated with the liver specific albumin promoter.

### DEFINITIONS

The following list defines terms and phrases used throughout the instant specification.

As used herein, the term “transgenic animal” means an animal having an exogenous gene (transgene) expressed in some or all of its cells, wherein the exogenous gene is introduced either by classical methods or by the methods of the instant invention.

As used herein, the term “transgene” refers to a nucleotide sequence that is artificially incorporated (either integrated or episomal) into the genome of a cell and is used to modify the cell for the purpose of achieving a genetic pool distinct from the “normal” or “wild-type” state. A transgene can be obtained from either the same cell (for example, but not limited to, a mutated gene) as the cell into which it is incorporated or it can be obtained from any other source; for example, but not limited to, a different organism or an artificial sequence.

As used herein, the term “exogenous gene” refers to a transgene that has been obtained from a different organism or cell than the organism or cell into which it has been incorporated.

The terms “transgene” and “exogenous gene” are used interchangeably herein.

As used herein, the term “wild-type” refers to the natural organism or cell or to the natural state of an organism or cell which is unmodified by man.

As used herein, the term “natural gastrointestinal pathway” refers to the pathway taken by ingested nutrients in a human or in an animal.

As used herein, the term “genetic material” can refer to DNA, RNA, ribozymes, antisense RNA, hybrids of DNA/RNA, either single or double stranded, or combinations thereof.

As used herein, the term “chimera” refers to a transgenic animal which is heterozygous for a particular trait. Chimeric animals are bred to obtain transgenic animals which are homozygous for a particular trait.

As used herein, the term “protective agent” refers to an agent that protects genetic material from destruction as it passes through a natural gastrointestinal pathway, for example, but not limited to, collagen, chitosan, alginate and the like.

As used herein, the term “transporting agent” refers to a compound containing an amine group that is capable of coupling with the genetic material in a manner effective to produce widespread distribution and cellular uptake subsequent to passage via a natural gastrointestinal pathway, for example, but not limited to, a polypeptide.

As used herein, the abbreviation “PLL” refers to poly-L-lysine.

### DETAILED DESCRIPTION OF THE INVENTION

The primary objective of this invention is the production of transgenic animals by administration (either oral or rectal) of a transporting agent, exemplified as, but not limited to an amino acid carrier, e.g. poly-l-lysine, polyarginine and polyornithine, for the purpose of carrying a compound, which although not limited to DNA, will nevertheless be exemplified as such for purposes of illustration herein, through the gastrointestinal tract and enabling its widespread distribution and systemic and sustained expression throughout the body.

In order for the compound, e.g. DNA to be distributable via the gastrointestinal tract with the highest possible degree of efficacy, it should be protected from enzyme degradation and low pH as it passes through the stomach and small intestine. In a preferred embodiment, this is accomplished via the use of protective compounds, illustrative of which are alginate, gelatin (which is mainly collagen) and the like.

The role of alginate, gelatin and collagen in protecting the key formulation (DNA-amino acid complex) through the stomach is very important to ensure DNA integrity (thereby facilitating the achievement of delivery efficacy), but can also be accomplished with alternative formulations such as chitosan, methacrylate, or alternatively, one or more of the conventional oral delivery systems used by the pharmaceutical industry, e.g. degradable capsules and gels.

The present inventors have determined that uncoupled (“naked”) DNA, if adequately protected with gelatin (collagen) or the like, is also taken through the intestinal wall and expressed in certain tissues, but not all of the tissues. However, it is important to distinguish that in this case: a) the efficacy of the delivery and expression of naked DNA is extremely low and b) it is not long-lasting, which is agreement with attempts to perfect the oral delivery of DNA described in the prior art. Thus, while the instant inventors have achieved limited success absent effective coupling to a transporting agent, this remains a non-preferred embodiment of the instant invention. Additionally, while the preferred, and most efficacious gastrointestinal route is via oral delivery, rectal delivery is indeed contemplated by the instant inventors as an alternative route for administration via the gastrointestinal pathway.

As we have noted above, the encapsulation of DNA in alginate-poly-L-lysine microcapsules has already been described, however prior artisans failed to appreciate the importance of coupling the therapeutic agent with the transport agent, e.g. via electrostatic binding, in a manner effective to produce efficacious and widespread distribution and cellular uptake subsequent to passage via said natural gastrointestinal pathway. While we have exemplified an embodiment which utilizes electrostatic binding, preferably via the use of positively charged amino acids which bind to a negatively charged therapeutic agent such as DNA, alternative binding techniques are contemplated for use in the instant invention.

Any transport agent is deemed to be useful in the context of the instant invention provided it couples with a therapeutic agent in a manner effective to produce efficacious and widespread distribution and cellular uptake subsequent to passage via said natural gastrointestinal pathway. Alternative transport agents contemplated as being useful within the context of this invention may include, but are not limited to, amino acids having an altered electrical charge, chemically modified compounds or amino acids, or synthesized molecules having the requisite functional groupings to make advantageous use of the natural transport pathways described herein.

Prior artisans such as Aggarwal et al. (Canadian Journal of Veterinary Research 63:148-152, 1999) and Mathiowitz et al. (Nature, Vol 386, March 1997, Pp. 410-414) teach the use of biodegradable and biologically adhesive microspheres respectively, as a means for oral drug delivery of genetic material containing agents such as DNA. Neither of these artisans recognized or pursued the use of a transport agent as outlined by the instant invention, nor did they recognize the value of coupling a therapeutic agent thereto so as to facilitate the widespread, systemic and sustained delivery and expression which are hallmarks of the instant inventive concept. In contrast, while not achieving the desirable distribution, delivery, efficacy or expression, the prior artisans nevertheless required encapsulation of the therapeutic agent, a requirement which is overcome via the instantly taught invention.

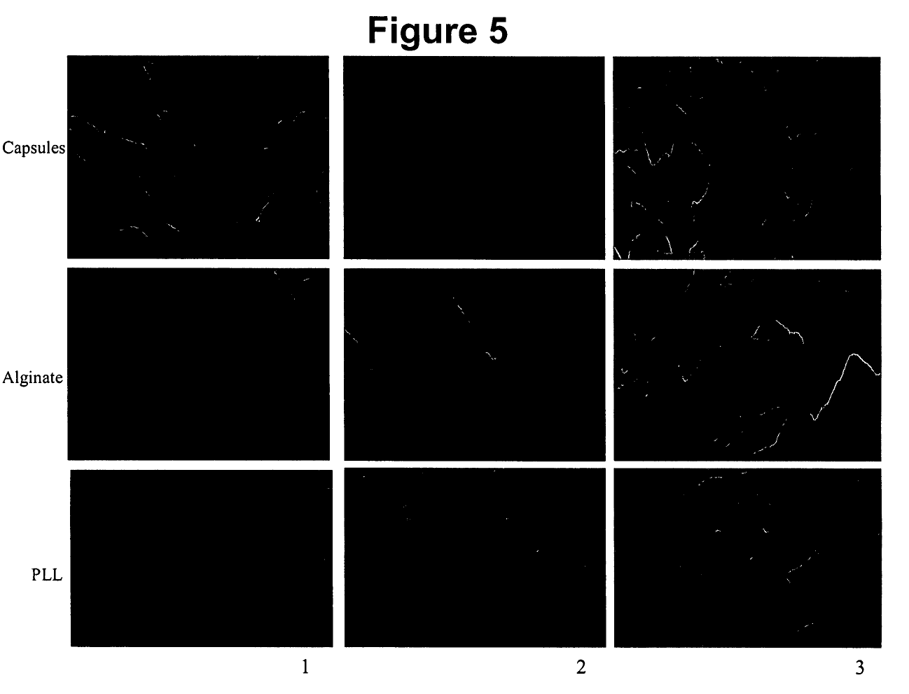

Mathiowitz et al. utilized polyanhydrides of a combination of fumaric and sebacic acids to encapsulate a plasmid DNA (β-galactosidase). However, as evidenced in FIG. 5 of the article, quantification of β-galactosidase activity in tissue extracts showed no significant activity in stomach or liver, but measurable activity within the intestine. This is indicative of an inability of the Mathiowitz technology to evidence transport through the intestine so as to enable delivery and/or expression in other organs.

In order to determine the relative effectiveness of the Aggarwal embodiments a comparative study was performed between a formulation in accordance with the instant invention (alginate-PLL-DNA) nanoparticles (hereafter referred to as alginate formulations) and the alginate-PLL microcapsules made by internal gelation as described in Aggarwal and hereafter referred to as Canola capsules (made using canola oil).

A single dose of 100 micrograms of a DNA plasmid containing the human growth hormone cDNA in an alginate-DNA-PLL nanoparticles in accordance with the instant invention was administered orally to C57BL/6 mice. A second group of mice (n=3) received the same plasmid in canola capsules. Note that these mice each received 300 micrograms of DNA, rather than the 100 micrograms given in the alginate formulation (three times more DNA). A control group of mice received nothing.

Mice were bled on days 0, 3 and 5 (so as to compare expression up to day 5, thus reproducing the results as determined by Aggarwal et al.).

The level of human growth hormone (hGH) in mouse serum on day 5 following the treatment was determined by ELISA (UBI Inc., NY).

Mice receiving alginate formulation had comparatively high levels of hGH in the serum. In contrast, hGH was not detected on day 5 in mice receiving canola capsules, even though mice receiving this formulation were administered three times more DNA than mice receiving the alginate formulation. As expected, control mice did not have detectable hGH in serum.

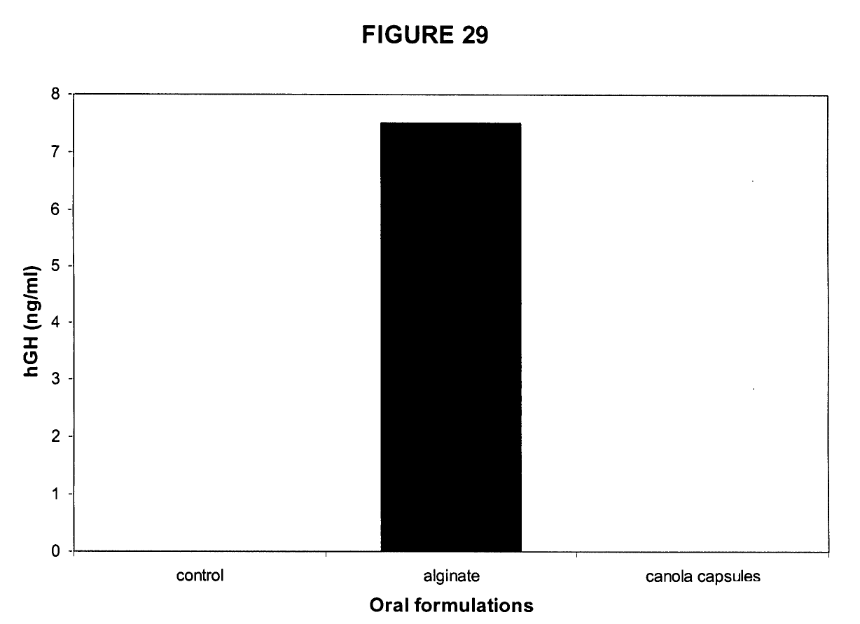

These data, as seen in FIG. 29 show that the efficacy of alginate formulation is much higher than canola capsules.

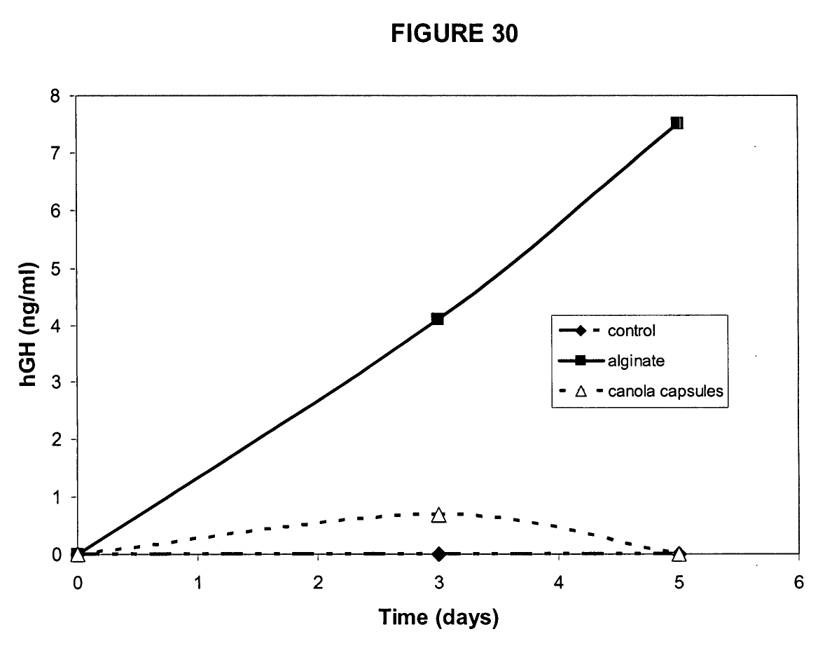

Now referring to FIG. 30, this graph depicts the level of hGH in mouse serum on days 3 and 5.

Mice administered Canola capsules had very modest but detectable hGH on day 3. However, this delivery was transient, and hGH was undetectable on day 5. This is consistent with the paper by Aggarwal et al., where it is necessary to feed mice daily for three days in order to detect circulating hGH on day 5. The transient nature of hGH delivery is consistent with the uptake of DNA by the intestine, rather than the distribution of DNA systemically, as taught by the instant invention.

In contrast, mice administered alginate formulation showed high hGH levels on day 3, that continue to increase on day 5. This is consistent with all our previous data, indicating that the alginate formulation leads to sustained, not transient, gene expression.

Thus, the uptake and expression of DNA is different with both formulations. The different trend of hGH delivery with both formulations would suggest that both formulations are taken by different routes and/or mechanism(s).

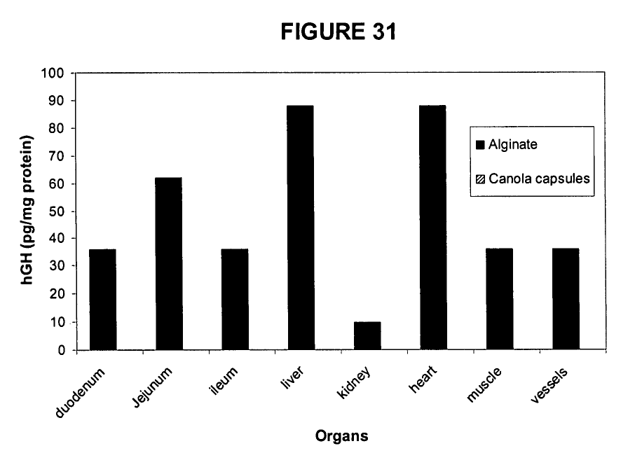

With reference to FIG. 31, on day 5, mice were sacrificed and the presence of hGH in the various organs was determined. High levels of hGH were recorded in the organs described in this graph in mice receiving alginate DNA formulation. In contrast, none of the mice receiving canola capsules had detectable hGH in any of the above organs, even though these mice received three times more DNA than the former group.

These results are consistent with our previous data showing wide systemic distribution of DNA in major organs following administration of alginate formulation. These results are also consistent with the lack of systemic distribution of DNA using formulations described in the prior art. Finally, these results also highlight the obvious difference in efficacy between both formulations.

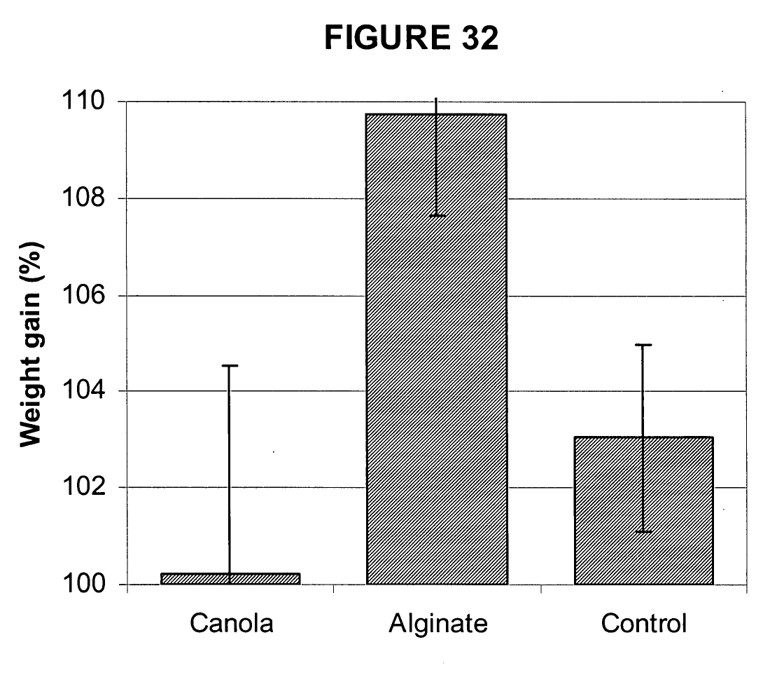

As further evidence of the efficacy of delivery in accordance with the present invention, a comparison of weight gain due to the presence of efficacious levels of hGH was determined and is the subject of FIG. 32.

It is known that the delivery of human growth hormone induces weight gain. However, gene therapy experiments delivering hGH have only demonstrated weight gain after very high levels of hGH are delivered (efficacious levels).

All mice were weighed on day 0, before treatment, and during the 5 days of the experiment. Mice that were fed canola capsules did not gain more weight than the control mice (p<0.145). In contrast, mice that were fed alginate formulation gained weight amounting to a 109.7% increase on day 5. The difference in weight gain between mice fed alginate formulation and mice receiving canola capsules was statistically significant (p<0.05).

The mice fed alginate formulation were considered to be transgenic for the expression of a human transgene (hGH) based on the systemic and sustained expression shown in these experiments.

Prior artisans have used DNA bound to PLL, but it has not been effective in delivering genes into animals because they failed to recognize the importance of oral delivery. Prior artisans have used orally administered DNA protected with chitosan, but failed to bind DNA to a transporting and distribution agent, such as polypeptides, thus failing to produce widespread distribution. Prior artisans have also used oral delivery of DNA (oligonucleotides—short segments of DNA—not including a whole gene or genetic regulatory sequences), enclosed in alginate-PLL microcapsules, albeit not coupled or conjugated to the transporting agent (as is required by the instant invention), with the intent of retrieving DNA from feces and thereby determining if DNA had mutated through the intestine. These artisans failed to recognize or suggest whether DNA could be taken up by the intestine and expressed, and therefore failed to recognize the instantly disclosed product or process of oral gene delivery. Oral delivery of DNA for widespread distribution, in conjunction with systemic and sustained expression of therapeutics has thus not heretofore been achieved.

Furthermore, in addition to DNA, it is contemplated to similarly transport additional therapeutic agents, non-limiting examples of which are RNA, which has commercial interest owing to its ability to inactivate the transcription/translation of unwanted proteins; and ribozymes, which are defined as catalytic RNA having the ability to recognize, bind and cleave a specific sequence of cellular RNA such as that of a virus, which could be delivered as a means of treating infectious diseases, such as AIDS.

DNA Microcapsules:

In the formation of the various species of the invention as hereafter described, it is understood that those molecules useful as transporting agents will exhibit the ability to form charged molecules, e.g. positive or negative side chains, so as to enable binding, e.g. conjugation, of the active agent with the transporting agent.

### DNA MICROCAPSULES—EXAMPLE 1

In a particular, albeit non-limiting embodiment, formation of DNA plasmids containing a cDNA coding for a transgene and appropriate genetic regulatory elements such as a promoter is performed as follows. A suspension of DNA is mixed with 1.5% potassium alginate (Kelmar, Kelco Inc., Chicago, USA) in a syringe and extruded through a 27 G needle with a syringe pump (39.3 ml/h). An air-jet concentric to the needle created fine droplets of the DNA/alginate mixture that are collected in a 1.1% CaCl₂ solution. Upon contact, the alginate/DNA droplets gel. After the microcapsules are extruded, they are subjected to the washes as indicated in the list below. The outer alginate layer is chemically cross-linked with poly-L-lysine hydrobromide (PLL, Sigma, St. Louis, USA) with Mr in a 15,000-30,000 range for 6 minutes, and then with another layer of alginate. Finally, the remaining free alginate core may be dissolved with sodium citrate for 3 minutes, to yield microcapsules with an alginate-PLL-alginate membrane containing DNA inside. The standard microcapsule protocol uses a 6 minutes citrate wash. With 3 minutes of citrate we increase the concentration of alginate left in the capsule core. This procedure appears to have an effect on the coupling of DNA.

Washes (unless stated otherwise, washing steps are performed with no incubation time in between):
1.1% calcium chloride
0.55% calcium chloride
0.28% calcium chloride
0.1% CHES (2-(Cyclohexylamino)ethanesulfonic acid) for about 3 minutes
1.1% calcium chloride
0.05% PLL for about 6 minutes
0.1% CHES (2-(Cyclohexylamino)ethanesulfonic acid)
1.1% calcium chloride
0.9% sodium chloride
0.03% potassium alginate for about 4 minutes
0.9% sodium chloride
0.055 M sodium citrate for about 3 minutes (standard microcapsule protocol is 6 minutes)
0.9% sodium chloride

### DNA MICROCAPSULES—EXAMPLE 2

A volume of 300 μl of DNA plasmid at a concentration of 1 μg/μl is mixed with 6 ml of 1.5% calcium alginate. Alginate beads are cross-linked with, e.g. Poly-L-Lysine (PLL) resulting in microcapsules containing DNA-alginate in the inside. Microcapsules are subsequently mixed with a 1:1 volume of a 50% gelatin solution to obtain a homogeneous mixture that can be administered.

DNA-Alginate-PLL Particles:

A volume of 100 μl of DNA plasmid at a concentration of 1 μg/μl is mixed with 50 μl of 3% calcium alginate, and mixed at 4° C. for 3 hours with gentle agitation. A volume of 50 μl of poly-L-Lysine is added. The mixture is vortexed for 30 seconds and mixed at 4° C. for one additional hour with gentle agitation. Finally, 50 μl of a 50% gelatin solution is added to the mixture to obtain a homogeneous mixture that can be administered.

DNA-PLL-Alginate Particles:

In an exemplary, but non-limiting example of forming DNA-PLL-Alginate microcapsules, a volume of 100 μl of DNA plasmid at a concentration of 1 μg/μl is mixed with 50 μl of poly-L-Lysine, and mixed at 4° C. for 3 hours with gentle agitation. A volume of 50 μl of 3% calcium alginate is added. The mixture is vortexed for 30 seconds and mixed at 4° C. for one additional hour with gentle agitation. Finally, 50 μl of a 50% gelatin solution is added to the mixture to obtain a homogeneous mixture that can be administered.

DNA-Ornithine-Alginate Particles:

A volume of 100 μl of DNA plasmid at a concentration of 1 μg/μl is mixed with 50 μl of poly-L-Ornithine. The mixture is vortexed for 30 seconds and mixed at 4° C. for 3 hours with gentle agitation. A volume of 50 μl of 3% calcium alginate is added and mixed at 4° C. for one additional hour with gentle agitation. Finally, 50 μl of a 50% gelatin solution is added to the mixture to obtain a homogeneous mixture that can be administered.

DNA-Arginine-Alginate Particles:

A volume of 100 μl of DNA plasmid at a concentration of 1 μg/μl is mixed with 50 μl of poly-L-Arginine. The mixture is vortexed for 30 seconds and mixed at 4° C. for 3 hours with gentle agitation. A volume of 50 μl of 3% calcium alginate is added and mixed at 4° C. for one additional hour with gentle agitation. Finally, 50 μl of a 50% gelatin solution is added to the mixture to obtain a homogeneous mixture that can be administered.

Naked DNA in Collagen:

A volume of 100 μl of DNA plasmid at a concentration of 1 μg/μl is mixed with 50 μl of a 50% gelatin solution, and mixed thoroughly to obtain a homogeneous mixture that can be administered.

The formulations of the instant invention may also be manufactured as nanoparticles or macroparticles of a variety of sizes, in combination with amphiphilic compounds, or the like, so as to deliver a compound such as DNA coupled to an amino acid.

Although lysine, arginine and ornithine are illustrated herein as exemplary transporting agents, other compounds and/or compositions having at least the requisite functional groups and if required, an appropriate charge, may also function as transporting agents in a similar fashion.

The inclusion of particular genetic regulatory elements (promoters), afford the compositions of the instant invention the added utility of controllable expression in vivo. Tissue-specific expression of therapeutic genes can be achieved by using tissue-specific genetic regulatory elements that restrict gene expression to specific tissues. Via the judicious use of such promoters, the degree of expression may be tailored to meet specific needs.

For example, via the use of β-Actin, a ubiquitous promoter, widespread expression is achieved. Alternatively, use of tissue specific genetic regulatory elements, illustrated, but not limited to albumin promoter (liver expression), muscle creatine kinase (MCK) for muscle expression, and keratinocyte (skin expression) provide the ability to express protein in a particularly desired location, e.g. a specific portion of the body, specific organ, or specific cell or tissue type.

In accordance with the present invention administration of the DNA formulation can be either oral or rectal and can be administered to a “wild-type” or a transgenic animal, for example, the animal to which the formulation is administered can be genetically altered (transgenic, either classically or in accordance with the teachings of this invention) at the time of administration in order that the formulation can correct the previous genetic alteration, or alternatively provide further modification. The formulation can also be administered to a “wild-type” animal, for example, an animal that is genetically unmodified by man at the time of administration.

In accordance with the present invention a therapeutic agent includes any genetic material which is introduced into a host in order to instigate a desirable biological effect. Such genetic materials may include, but are not limited to DNA, RNA, Ribozyme, Antisense, Hybrids, either Single or Double stranded, or combinations thereof.

In accordance with the present invention a desirable biological effect may include, but is not limited to, gene expression, gene inhibition, and gene correction. Said biological effect may include, but is not limited to, those effects which are directly related to the cellular uptake of a therapeutic agent following oral delivery, e.g. FIX (Factor IX) DNA which leads to FIX production. Said biological effect may directly occur as a result of said cellular uptake, as a result of systemic expression, or alternatively targeted expression, which is understood to include expression specifically directed to a particular organ, system or a targeted cell or group of cells. Said biological effect is exemplified by, but not limited to, modulation of a disease state, wherein expression of a therapeutic agent modifies the onset, course, manifestation or severity of the disease state.

In accordance with the present invention systemic expression is understood to mean measurable cellular uptake of a therapeutic agent within cells, inclusive of, but not limited to cells of the epithelial, connective, nervous and musculo-skeletal tissues, found in various organs throughout the body.

In accordance with the present invention, sustained expression or sustained delivery is understood to mean measurable expression of a therapeutic agent sufficient to instigate a desirable biological effect, as a result of a single administration, which effect is detectable for a minimum of 40 days. The protein encoded by the therapeutic agent may be intracellular or extracellular.

In accordance with the present invention widespread distribution is understood to mean distribution of a therapeutic agent to essentially all organs (as evidenced and exemplified in Tables 1 and 2 and the accompanying figures), including but not limited to the central nervous system, in particular to the brain, heart and bone marrow; such distribution effected, for example, via the basal membrane of the intestinal epithelium and beyond to multiple organ sites.

In its preferred embodiments, the instant invention is directed toward the formation of a distributable moiety, which moiety is formed by the coupling of a transporting agent and at least one genetic material in a manner effective to provide, via a natural gastrointestinal pathway (e.g. orally or rectally), for widespread distribution, systemic expression and sustained delivery of said material. Said genetic material may, for example, be a complete transcriptional unit, which is broadly defined as the combination of at least a particular portion of DNA coding for a therapeutic agent for which expression is desired, in combination with a promoter and other genetic regulatory elements sufficient to provide expression, subsequent to intracellular absorption, of the desired therapeutic agent. Said agent may comprise any expressed entity which exhibits therapeutic value, and may include, but is not limited to, proteins, antibodies, DNA, RNA, or particular portions or fragments thereof.

While the use of a promoter for the expression of the transgene is considered to be mandatory in order to successfully accomplish systemic expression, (which is a hallmark of the present invention) a promoter is not mandatory when the goal is inhibition of the production of an existing therapeutic product (i.e. hepatitis virus or HIV genes in humans). Additionally, use of a tissue specific, as opposed to a ubiquitous promoter provides a degree of freedom in tailoring the degree of systemic expression achieved. Furthermore, delivery of antisense nucleic acids (RNA and/or DNA) or ribozymes may be accomplished without including a promoter.

Another application contemplated by the present technology, in which a complete transcriptional unit is not required, has to do with judicious utilization of inteins and exteins in order to achieve a type of gene therapy.

Inteins are insertion sequences embedded within a precursor protein, and they are capable of protein splicing that removes the intein sequence and at the same time ligates the flanking polypeptides (termed exteins). The therapeutic gene can be split into 2 distinct entities that are administered separately via the instantly disclosed technique.

Inteins have been utilized to produce a functional protein, following the splitting of the gene in two parts, that were expressed separately. After the two proteins are made (translation), the intein portions are removed (by themselves), and the adjacent extein portions (one at the end of a first part of the gene and the second at the beginning of second part of the gene part) are joined together to form a full functional protein.

The incorporation of a promoter within one portion will nevertheless be in order for both parts of the protein to be expressed.

Additionally, some vectors, such as

Adenoassociated-virus (AAV) form concatamers inside the infected cells. In the process the vector multiplies itself to create a series of copies of the vector that are placed one after the other. One can exploit this fact, using the instantly disclosed transport agent technology, to split a gene in half, and express both portions separately in two vectors. If one then transports and introduces both vectors inside the same cell, both vectors can come together physically, and the full promoter-gene context can be re-established inside the cell. Alternatively, as shown by Zhou et al. (“Concatamerization Of Adeno-Associated Virus Circular Genomes Occurs Through Intermolecular Recombination” J Virology 1999 November; 73(11):9468-77), one could place the promoter in one vector, and the transgene in a second vector, that are administered separately.

The following listing of amino acids, their derivatives, and related compounds, are non-limiting illustrative examples of compounds containing the requisite structure deemed necessary for widespread distribution of DNA in vivo.

Amino Acids and Derivatives:

Aliphatic—alanine, glycine, isoleucine, leucine, proline, valine
Aromatic—phenylalanine, tryptophan, tyrosine
Acidic—aspartic acid, glutamic acid
Basic—arginine, histidine, lysine
Hydroxylic—serine, threonine
Sulphur-containing—cysteine, methionine
Amidic (containing amide group)—asparagine, glutamine
   Peptides:

Two individual amino acids can be linked to form a larger molecule, with the loss of a water molecule as a by-product of the reaction. The newly created C—N bond between the two separate amino acids is called a peptide bond. The term ‘peptide bond’ implies the existence of the peptide group which is commonly written in text as —CONH—;
Dipeptide: two molecules linked by a peptide bond become what is called a dipeptide;
Polypeptide: a chain of molecules linked by peptide bonds;
Proteins: made up of one or more polypeptide chains, each of which consists of amino acids which have been mentioned earlier.

It is known that when a living cell makes protein, the carboxyl group of one amino acid is linked to the amino group of another to form a peptide bond. The carboxyl group of the second amino acid is similarly linked to the amino group of a third, and so on, until a long chain is produced, called a polypeptide. A protein may be formed of a single polypeptide chain, or it may consist of several such chains held together by weak molecular bonds. The R groups of the amino acid subunits determine the final shape of the protein and its chemical properties; whereby an extraordinary variety of proteins are produced. In addition to the amino acids that form proteins, more than 150 other amino acids have been found in nature, including some that have the carboxyl and amino groups attached to separate carbon atoms. These unusually structured amino acids are most often found in fungi and higher plants. Any having the requisite functional groupings, and which are capable of being coupled to the therapeutic agent of choice are contemplated for use within the instant invention.

As used herein, the term Deoxyribonucleic acid (DNA) is understood to mean a long polymer of nucleotides joined by phosphate groups, DNA is the genetic material that provides the blueprint for the proteins that each different cell will produce in its lifetime. It consists of a double stranded helix consisting of a five-sided sugar (deoxyribose) without a free hydroxyl group, a phosphate group linking the two nucleotides, and a nitrogenous base.

As used herein, the term Ribonucleic acid (RNA) is understood to mean a long polymer of ribose (a five-sided sugar with a free hydroxyl group) and nitrogenous bases linked via phosphate groups. It is complementary to one of the DNA strands and forms the proteins that are specified by the cell.

As used herein the term Zwitterions is understood to mean amino acids in a form of neutrality where the carboxyl group and amino group are ready to donate and accept protons, respectively.

The evolution and mutation of proteins can be realized through changes in deoxyribonucleic acid (DNA). DNA is translated to proteins via ribonucleic acid (RNA). Although every cell contains an identical copy of DNA with complete instructions for all types of body tissues, only certain proteins are produced by each cell type. In this way, cells of different tissues can perform diverse tasks through the production of unique proteins. In accordance with the teachings of the present invention, a therapeutic agent, e.g. DNA or RNA may be generally distributed throughout an organism via oral administration, thereby eliciting a detectable alteration. This detectable alteration may be broadly directed toward all cells of the organism, thereby effecting a cure for a disease, or enhancement of a particular characteristic.

Alternatively, by judicious use of organ or tissue specific promoters, the detectable alterations may be limited to expression in particularly determined locations, thereby providing a safe and effective means for oral administration of chemical or genetic modifiers, whose locus of activity is particularly controlled.

The amino acids that form charged side chains in solution are lysine, arginine, histidine, aspartic acid, and glutamic acid. While aspartic acid and glutamic acid release their protons to become negatively charged in normal human physiologic conditions, lysine and arginine gain protons in solution to become positively charged. Histidine is unique because it can form either basic or acidic side chains since the pKa of the compound is close to the pH of the body. As the pH begins to exceed the pKa of the molecule, the equilibrium between its neutral and acidic forms begins to favor the acidic form (deprotonated form) of the amino acid side chain. In other words, a proton is more likely to be released into solution. In the case of histidine, a proton can be released to expose a basic NH2 group when the pH rises above its pKa (6). However, histidine can become positively charged under conditions where the pH falls below 6. Because histidine is able to act as an acid or a base in relatively neutral conditions, it is found in the active sites of many enzymes that require a certain pH to catalyze reactions, and is contemplated as being useful in the instant invention.

Amino acids can be polar or non-polar. Polar amino acids have R groups that do not ionize in solution but are quite soluble in water due to their polar character. They are also known as hydrophilic, or “water loving” amino acids. These include serine, threonine, asparagine, glutamine, tyrosine, and cysteine. The nonpolar amino acids include glycine, alanine, valine, leucine, isoleucine, methionine, proline, phenylalanine and tryptophan. Nonpolar amino acids are soluble in nonpolar environments such as cell membranes and are called hydrophobic molecules because of their “water fearing” properties. These compounds are contemplated for use where a charge may be induced or wherein the therapeutic agent is caused to be charged so as to initiate a coupling effect.

### a. EXAMPLES

Biodistribution of Oral DNA which Expresses Green Fluorescent Protein (GFP)

Single administration of alginate/PLL GFP DNA nanoparticles in mice (n=3) was carried out. Three formulations were tested:
1) DNA alginate/PLL microcapsules (Capsules);
2) Alginate/DNA/PLL nanoparticles (Alginate); and
3) PLL/DNA/alginate nanoparticles (PLL).

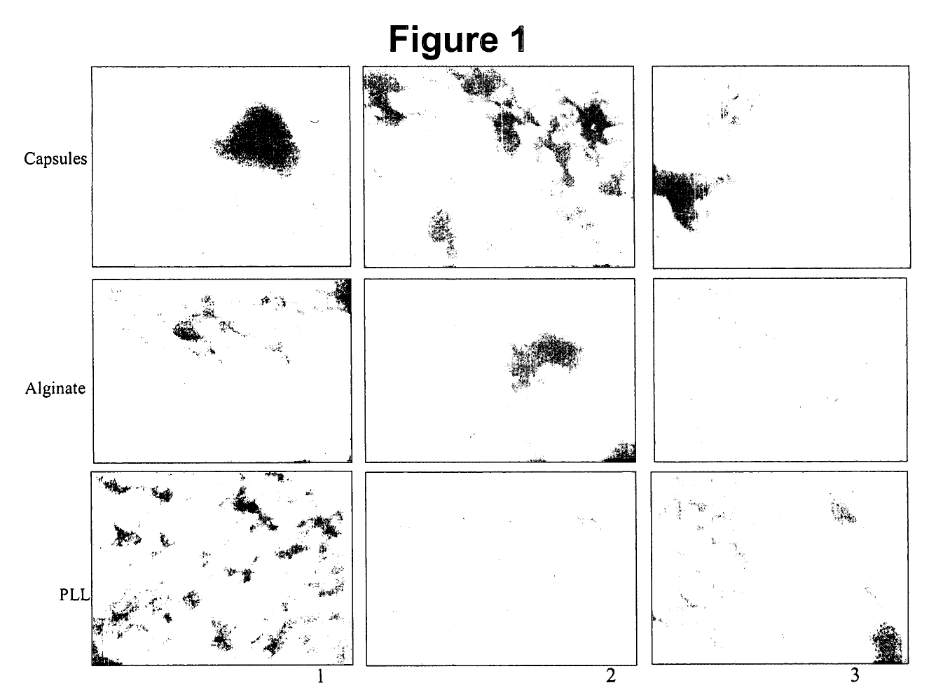

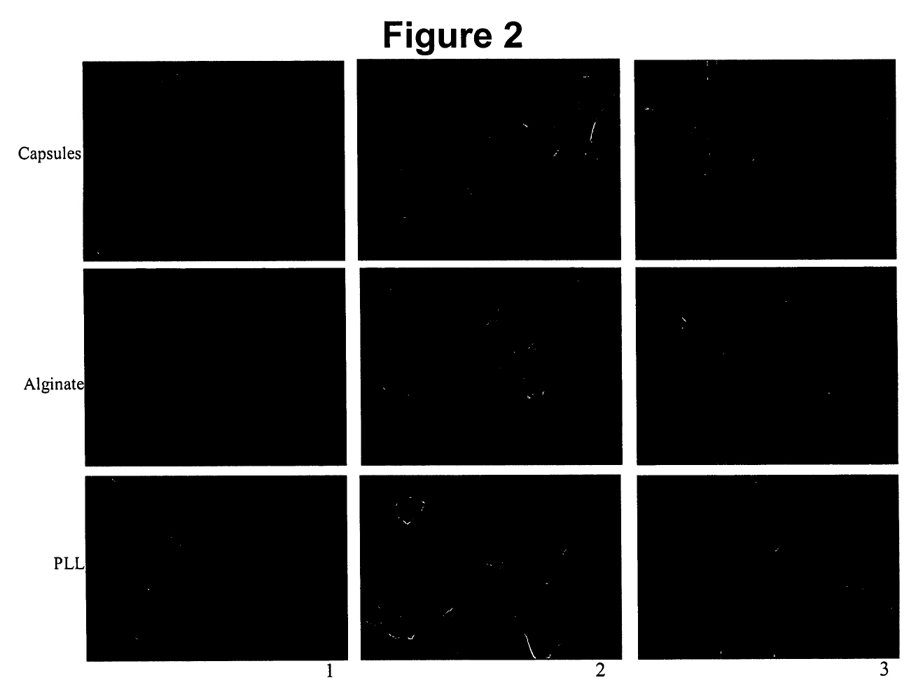

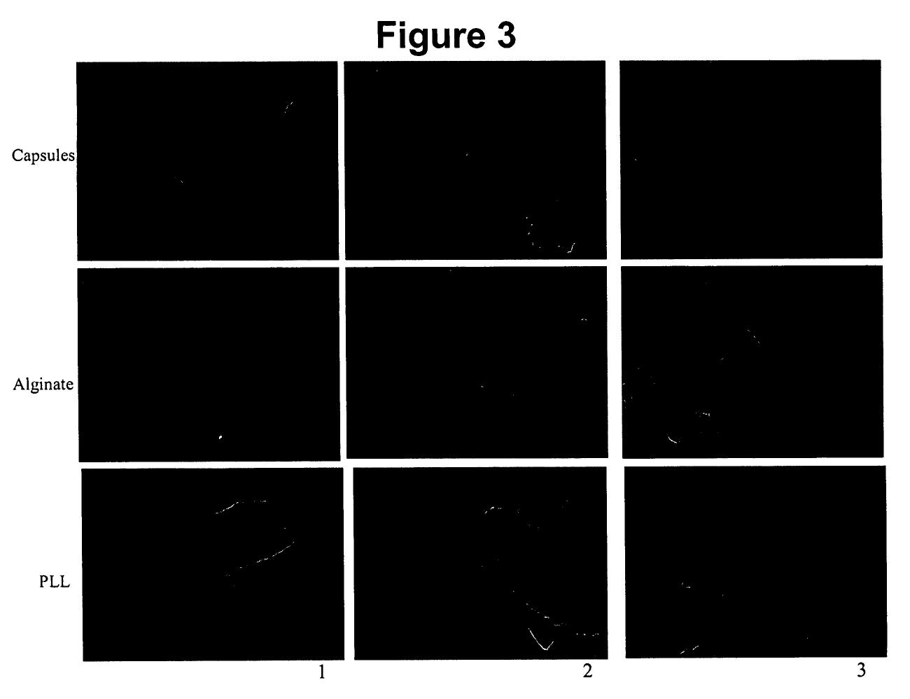

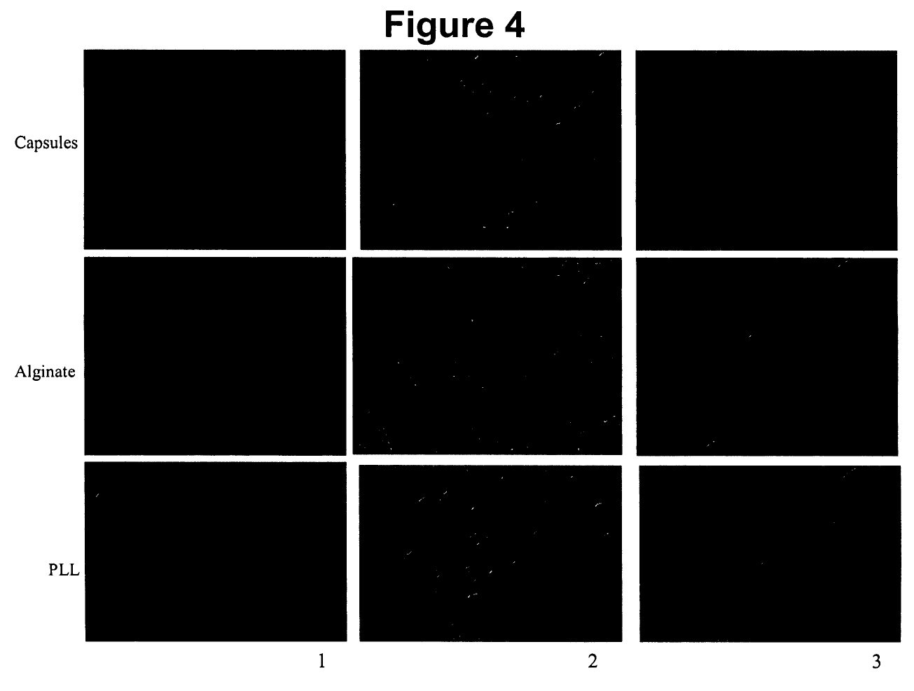

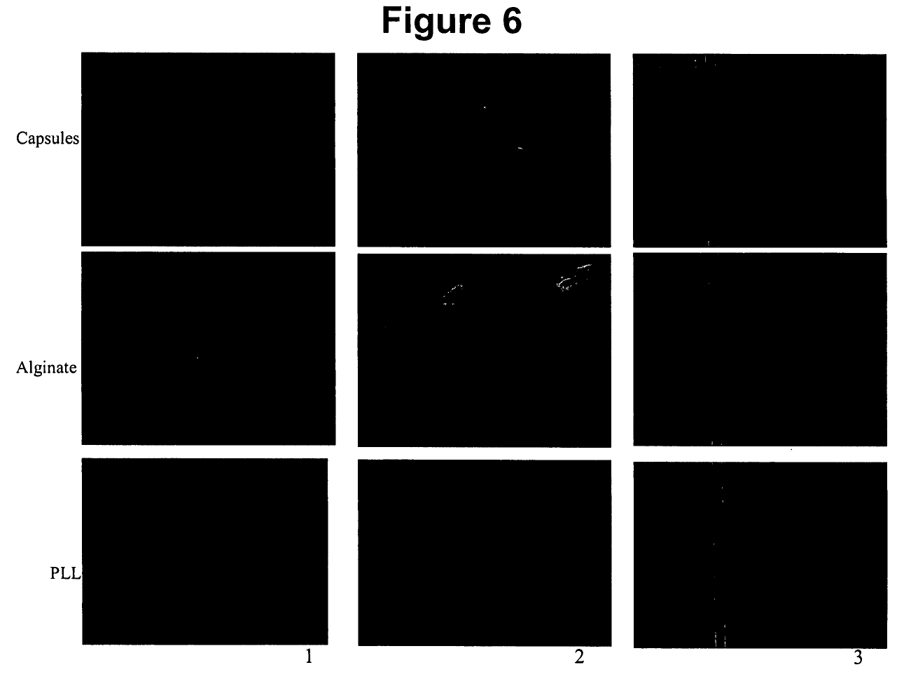

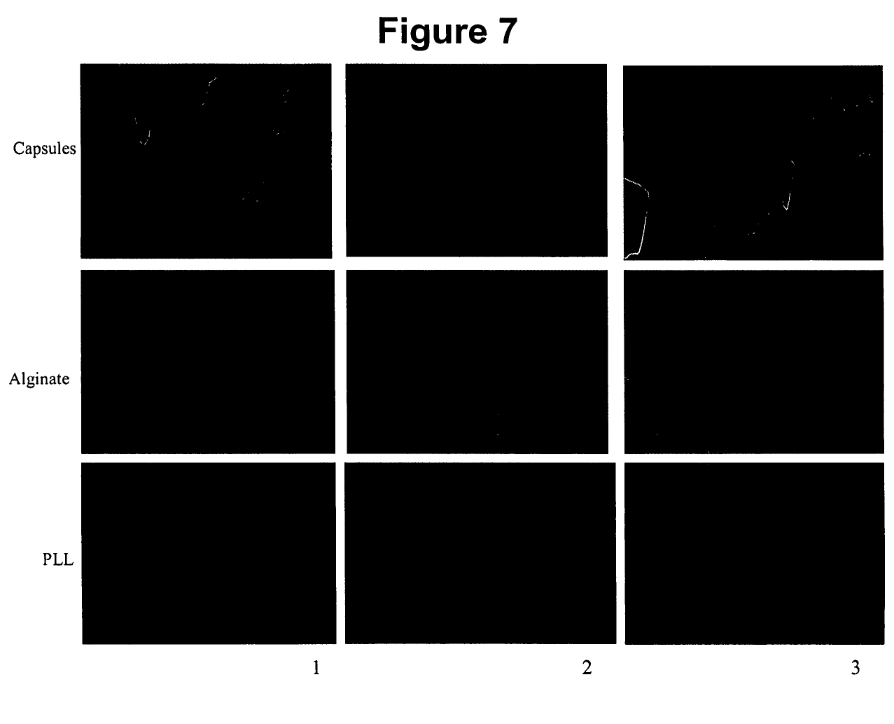

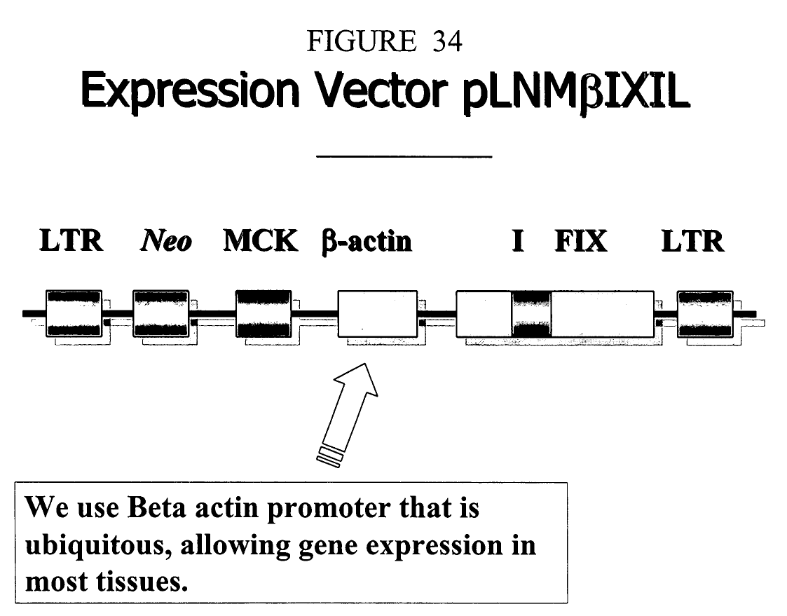

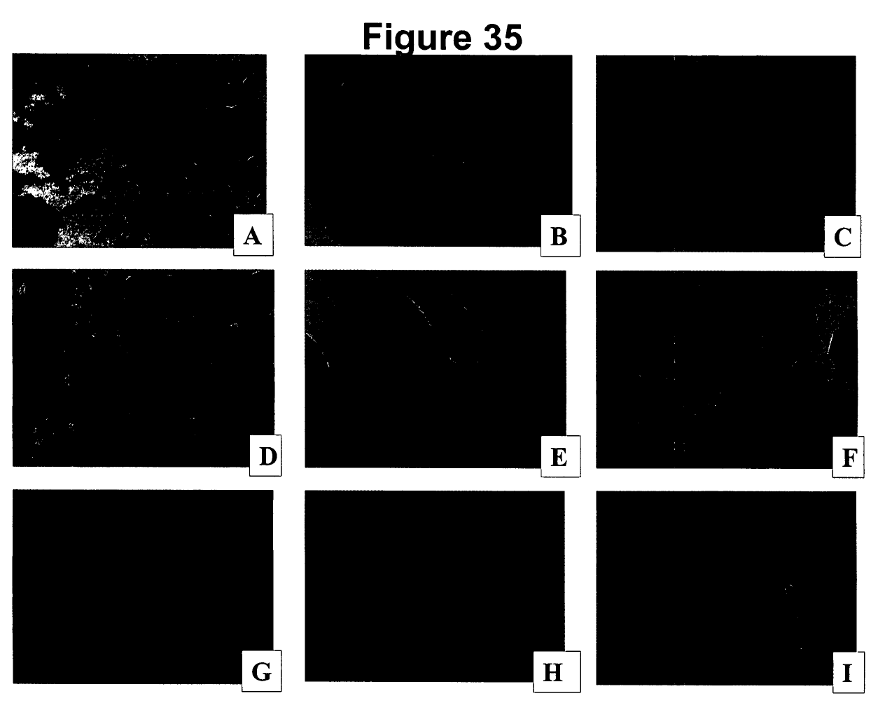

9 mice were treated, and were sacrificed on Day 42. Tissue samples from all are illustrated in fluorescent micrographs designated as FIGS. 1-7. FIG. 1 is a fluorescent micrograph illustrating expression in the Liver; FIG. 2 is a fluorescent micrograph illustrating expression in the Kidney; FIG. 3 is a fluorescent micrograph illustrating expression in the Lung; FIG. 4 is a fluorescent micrograph illustrating expression in the Heart; FIG. 5 is a fluorescent micrograph illustrating expression in the Muscle; FIG. 6 is a fluorescent micrograph illustrating expression in the Skin; and FIG. 7 is a fluorescent micrograph illustrating expression in the Vessels. FIG. 34 illustrates a vector used for expression of GFP.

GFP (green fluorescent protein) is intracellular and stays in the cell where it is produced. As is readily apparent by reviewing the accompanying figures (especially FIG. 35) and as summarized in the Table 1, fluorescent microscopy detects virtually all cells in all major organs examined as being green.

TABLE 1

| | | | | | | | | Vessel |
|---|---|---|---|---|---|---|---|---|
| Tissue | Liver | Kidney | Lung | Heart | Muscle | Brain | Skin | (Aorta) |
| Capsules | +++ | ++ | ++ | +++ | +++ | ++ | +++ | +++ |
| Alginate | +++ | ++ | ++ | +++ | +++ | ++ | +++ | ++ |
| PLL | +++ | ++ | ++ | +++ | +++ | ++ | +++ | + |

| | Bone | | | | | | | |
|---|---|---|---|---|---|---|---|---|
| Tissue | marrow | Spleen | Pancreas | Duodenum | Jejunum | Ileum | Colon | Gonads |
| Capsules | ++ | ++ | ++ | ++ | +++ | +++ | + | + |
| Alginate | ++ | +++ | +++ | ++ | +++ | +++ | + | + |
| PLL | ++ | + | ++ | ++ | +++ | +++ | + | + |

This indicates that DNA, in the form of microcapsules conjugated with the transporting agent (PLL) and internalized within a capsule comprising cross-linked alginate/transporting agent goes through the intestine and is transported to all major organs where it enters the cells and is efficiently expressed. This is in contradistinction to prior art encapsulated DNA, wherein the PLL acted as a structural element which prevented/reduced diffusion of DNA.

As a validation of the technique, analysis of tissue samples was performed utilizing polymerase chain reaction (PCR) as an amplification technique.

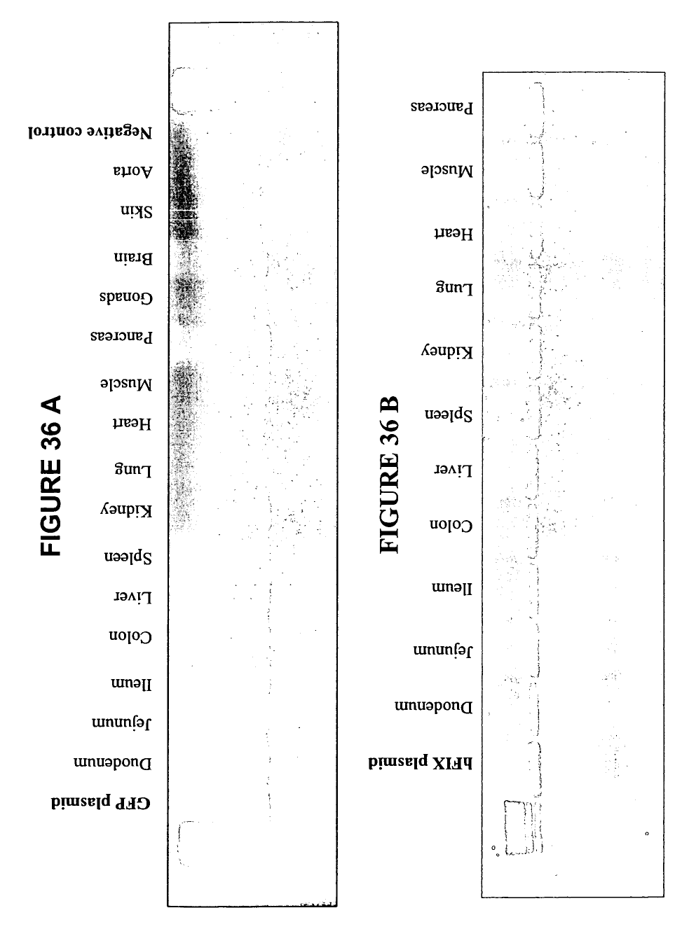

At day 42 post-treatment, the mice were sacrificed. DNA from various tissues was amplified by PCR and separated by agarose gel electrophoresis (FIG. 36A), and showed that orally administered DNA is found in every major organ examined (Table 2). This finding further confirms that DNA administered orally is taken to all organs, where it enters cells. These mice expressing GFP can be considered transgenic.

TABLE 2
PCR of GFP DNA in tissues (day 42)
Tissue

| Liver | Kidney | Lung | Heart | Muscle | Brain | Skin |
|---|---|---|---|---|---|---|
| Positive | Positive | Positive | Positive | Positive | Positive | Positive |

Tissue

| | | | | | | Vessel |
|---|---|---|---|---|---|---|
| Spleen | Pancreas | Duodenum | Jejunum | Ileum | Colon | (Aorta) |
| Positive | Positive | Positive | Positive | Positive | Positive | Positive |

Note:
PCR in bone marrow and gonads were not conducted.

1) Example

To determine the importance of alginate and PLL for efficient expression of oral DNA the following experiment was carried out.

A single administration of alginate/PLL hFIX (human Factor IX) DNA nanoparticles was given to mice (n=3). Three formulations were tested: DNA alginate/PLL nanoparticles (regular control), alginate/DNA nanoparticles (no PLL), and PLL/DNA nanoparticles (no alginate).

At day 3, 7 and 14 post-treatment, mice were bled. Control mice had hFIX in blood (approx. 70 ng/ml). None of the mice with no alginate or with no PLL had detectable hFIX (sensitivity 3 ng/ml). Thus, it was concluded that both alginate and PLL are needed to insure widespread DNA distribution and subsequent protein expression. While not wishing to be bound to a particular theory of operation, it appears that alginate protects DNA in the GI tract, and PLL helps distribute DNA into all organs.

1) Example Using HFIX

To determine the degree of expression obtainable, additional experimentation was conducted to demonstrate Human factor IX (FIX) delivery.

A single administration of alginate/PLL FIX DNA nanoparticles was carried out in hemophilic mice.

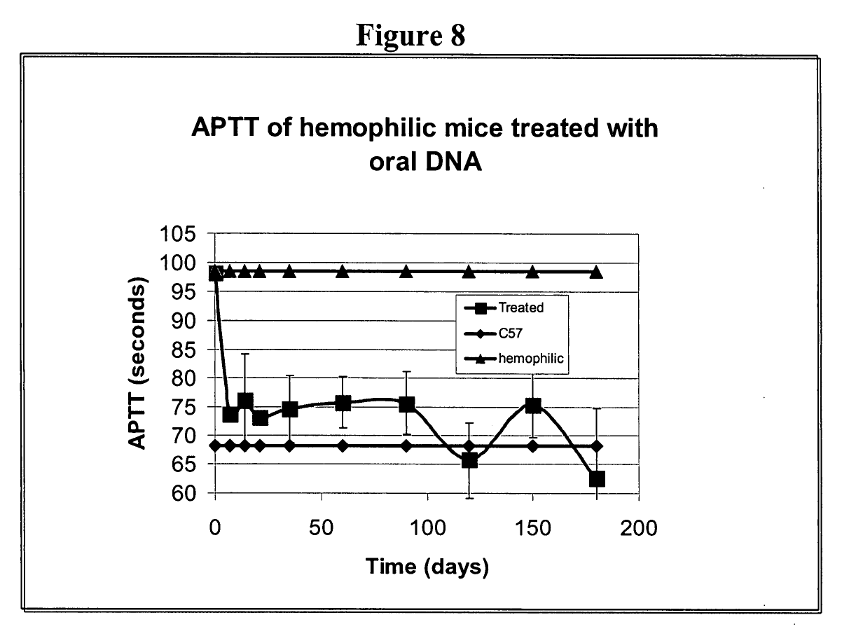

APTT (Blood clotting time test) was done to determine correction of the disease in the treated hemophilic mice. As further illustrated in FIG. 8, treated hemophilia mice demonstrated a normalized bleeding pattern for at least 180 days.

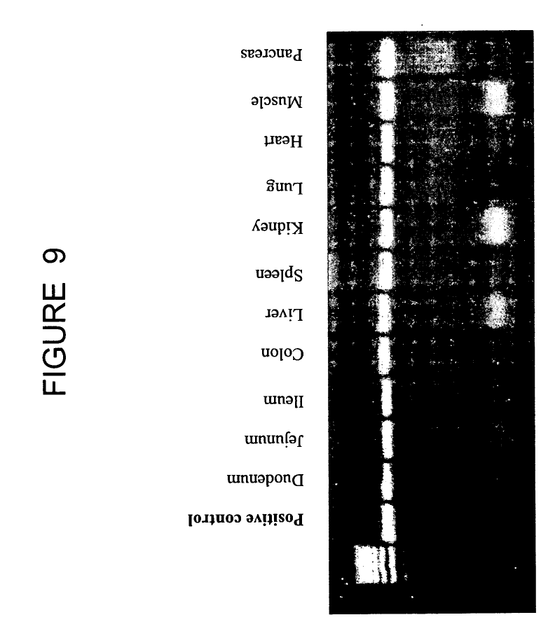

Now referring to FIG. 9, amplification of data via PCR was performed on tissue samples harvested from a plurality of organs on day 42 post ingestion of alginate/PLL GFP DNA nanoparticles. All organ samples demonstrated a positive presence of GFP via PCR analysis. This data is additionally set forth in Table 2 above.

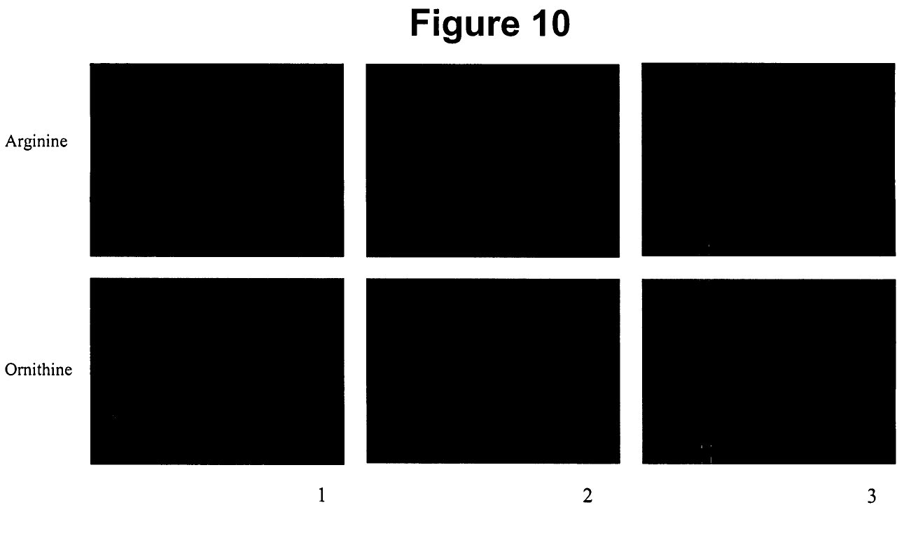

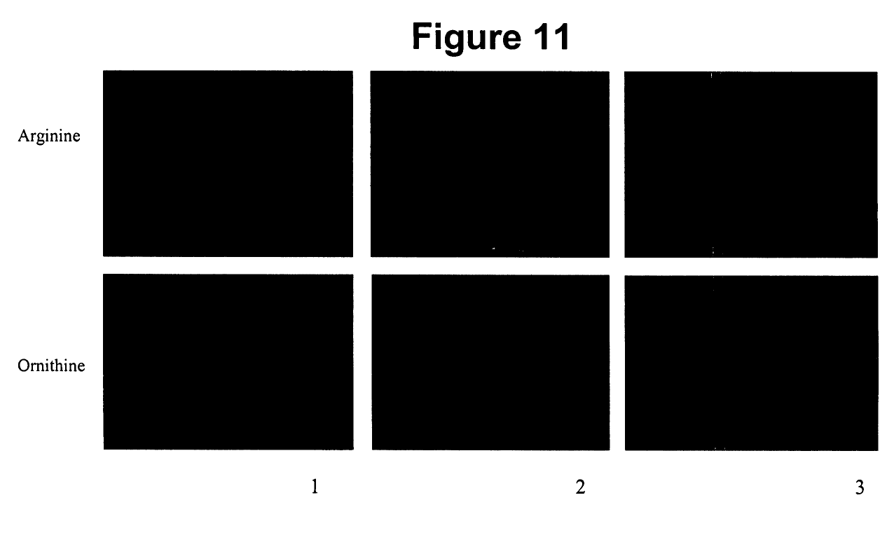

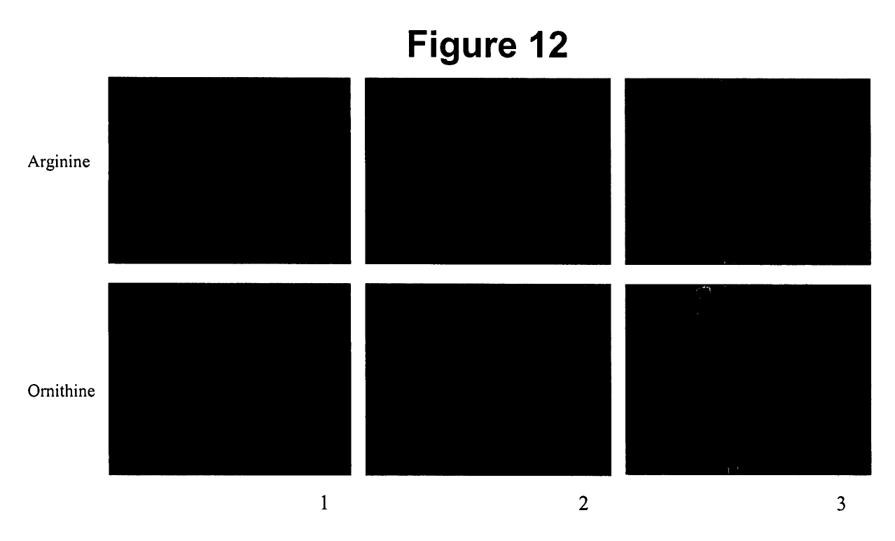

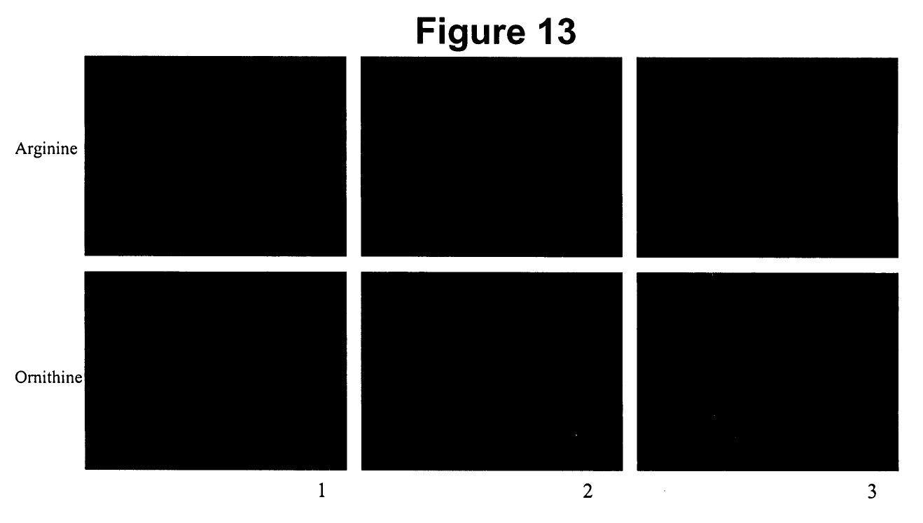

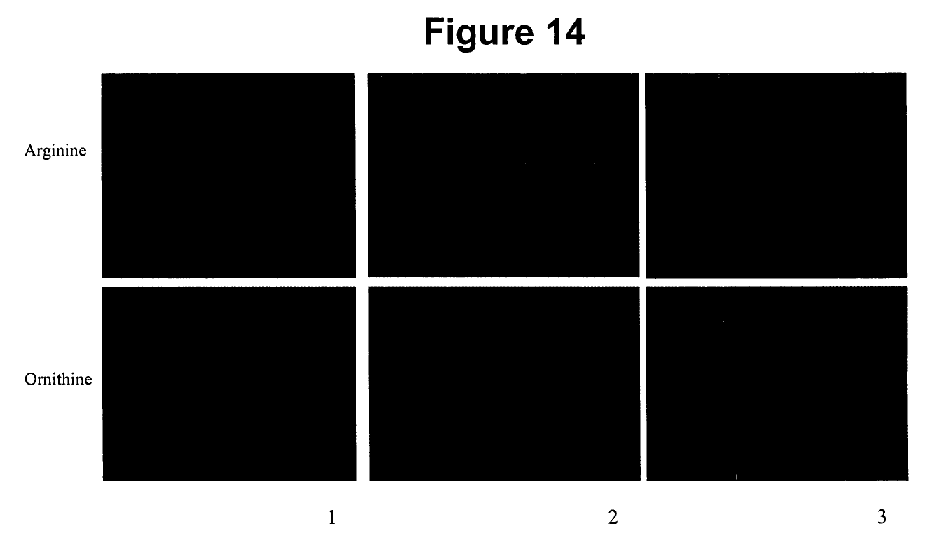

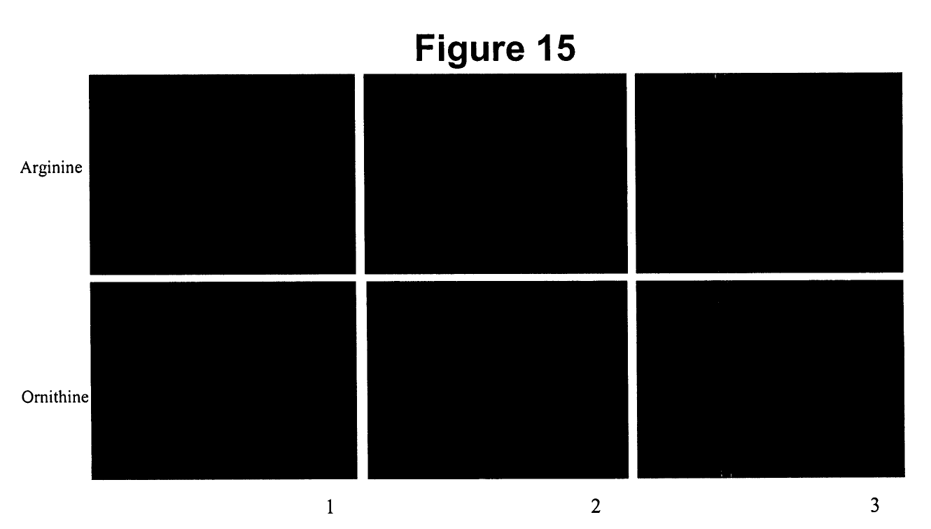

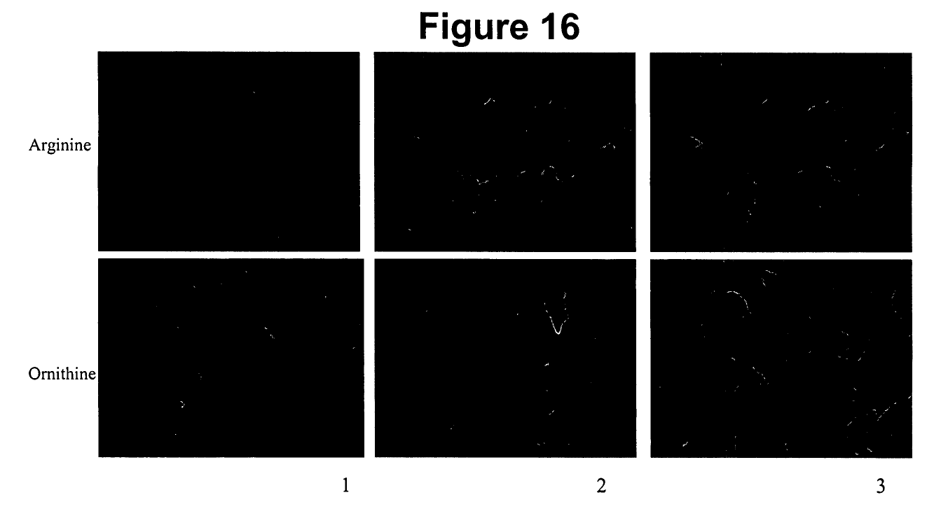

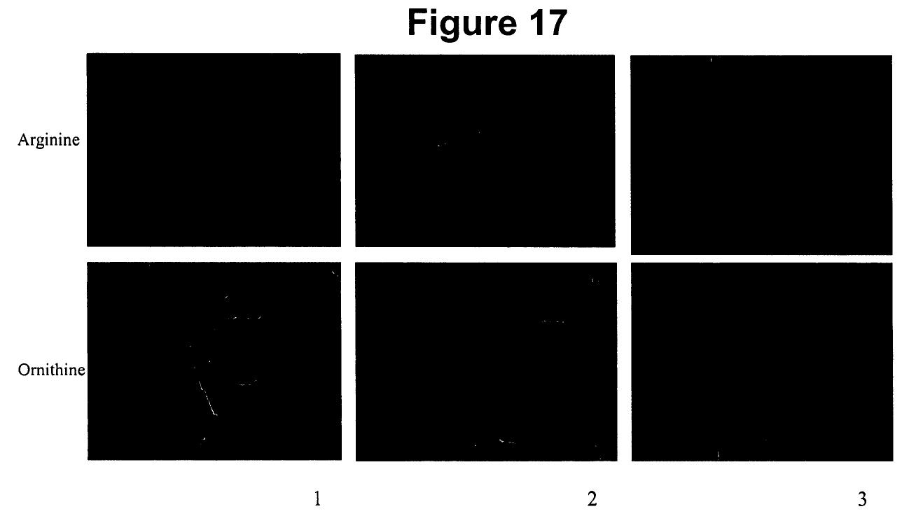

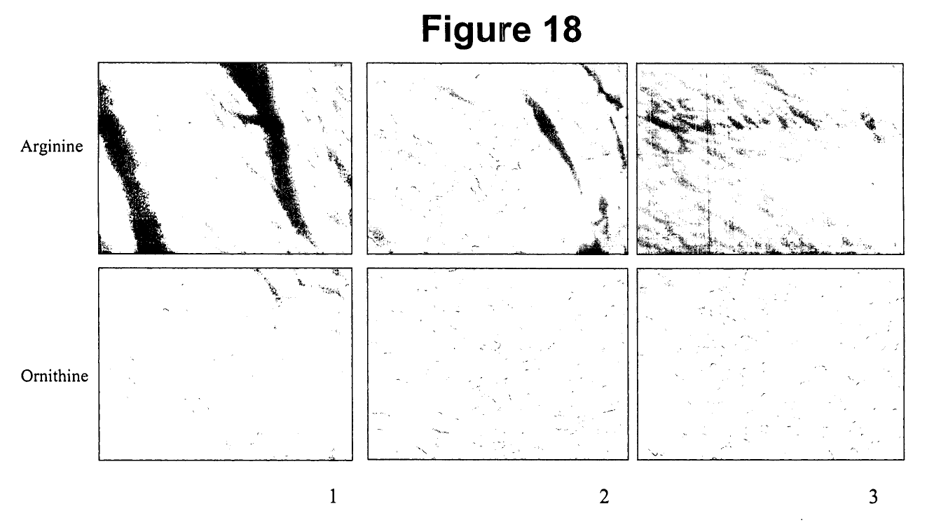

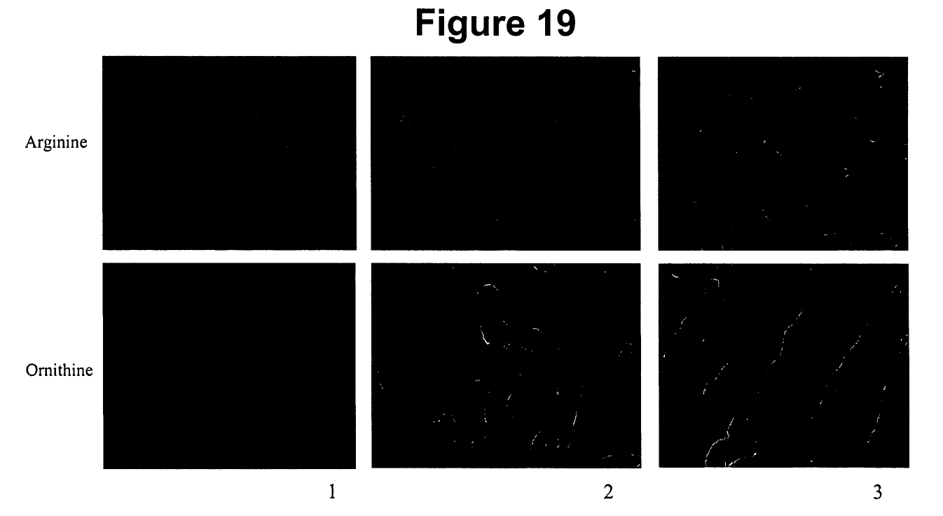

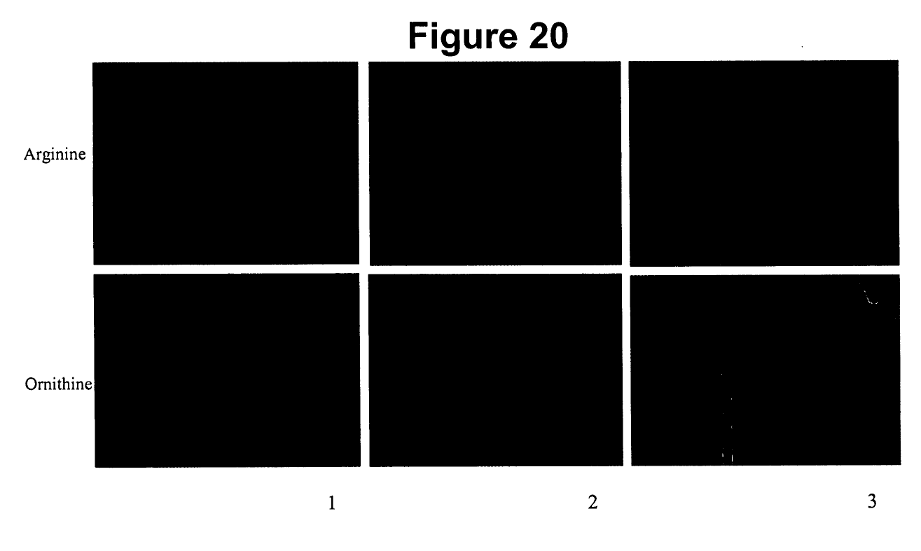

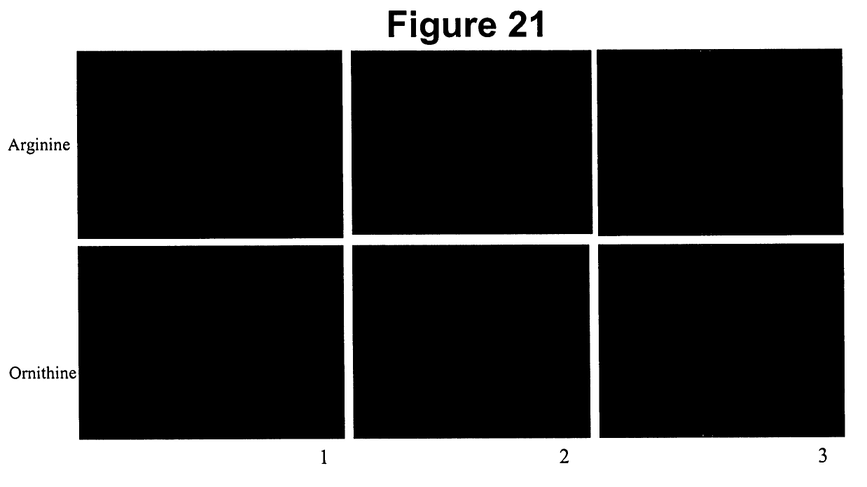

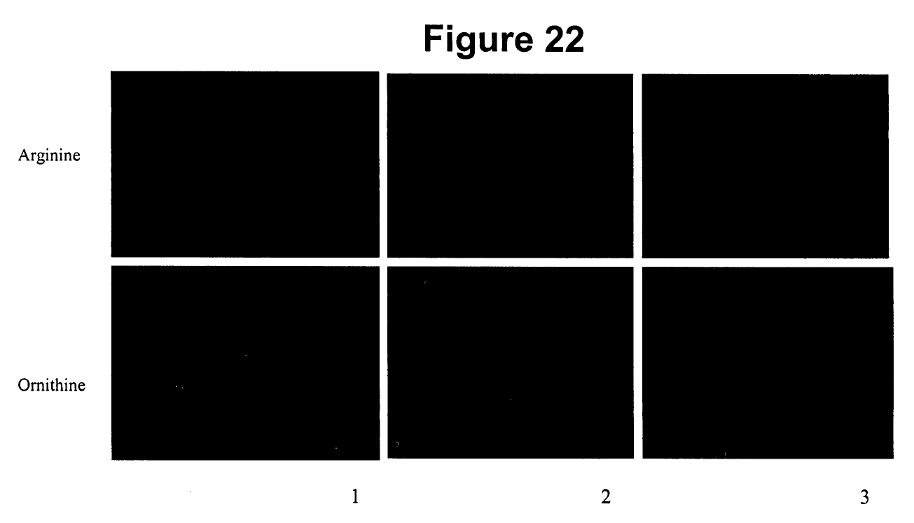

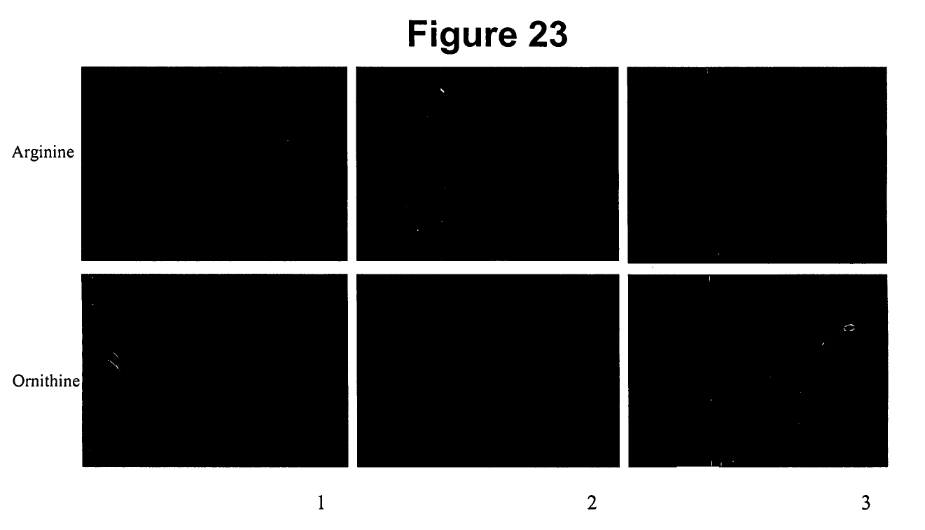

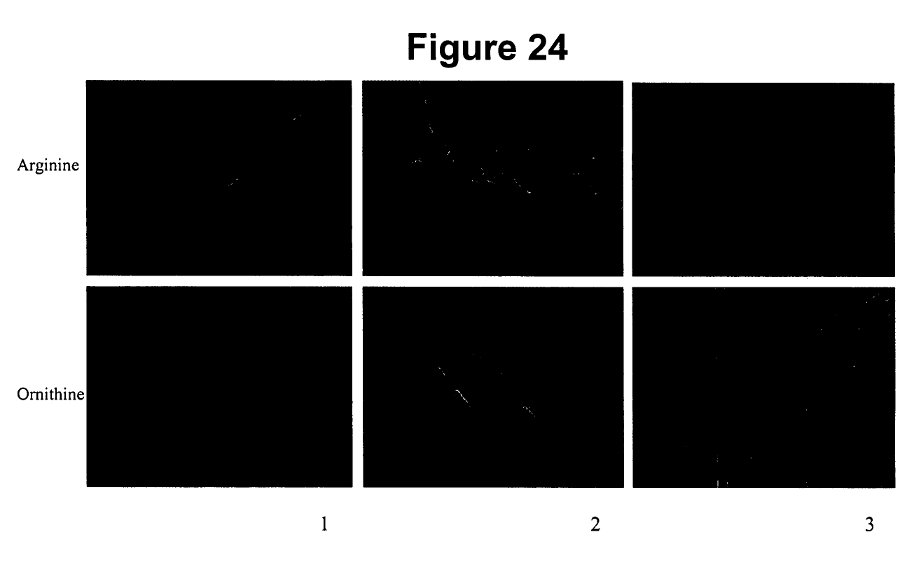

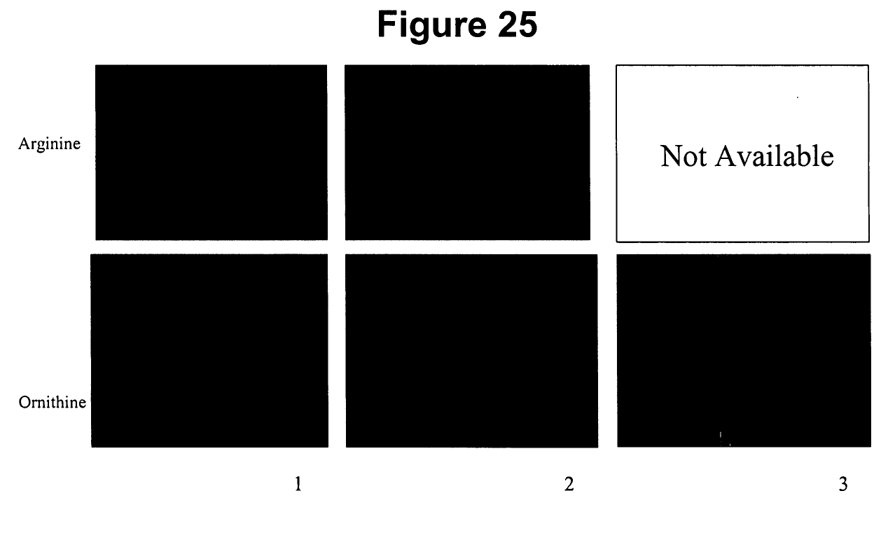

Further experimentation was conducted to validate the efficacy of distribution and expression using alternative transport agents. Poly-ornithine and poly-arginine were conjugated with DNA coding for GFP and alginate and formulated into nanoparticles. The nanoparticles were administered to mice (n=3) in a manner as earlier described. At day 10, the mice were sacrificed and fluorescent micrographs were taken (FIGS. 10-25). FIG. 10 is a fluorescent micrograph illustrating expression utilizing Arginine/Ornithine transport agents in the Duodenum; FIG. 11 is a fluorescent micrograph illustrating expression utilizing Arginine/Ornithine transport agents in the Jejunum; FIG. 12 is a fluorescent micrograph illustrating expression utilizing Arginine/Ornithine transport agents in the Ileum; FIG. 13 is a fluorescent micrograph illustrating expression utilizing Arginine/Ornithine transport agents in the Colon; FIG. 14 is a fluorescent micrograph illustrating expression utilizing Arginine/Ornithine transport agents in the Liver; FIG. 15 is a fluorescent micrograph illustrating expression utilizing Arginine/Ornithine transport agents in the Spleen; FIG. 16 is a fluorescent micrograph illustrating expression utilizing Arginine/Ornithine transport agents in the Kidney; FIG. 17 is a fluorescent micrograph illustrating expression utilizing Arginine/Ornithine transport agents in the Lung; FIG. 18 is a fluorescent micrograph illustrating expression utilizing Arginine/Ornithine transport agents in the Heart; FIG. 19 is a fluorescent micrograph illustrating expression utilizing Arginine/Ornithine transport agents in the Muscle; FIG. 20 is a fluorescent micrograph illustrating expression utilizing Arginine/Ornithine transport agents in the Pancreas; FIG. 21 is a fluorescent micrograph illustrating expression utilizing Arginine/Ornithine transport agents in the Brain; FIG. 22 is a fluorescent micrograph illustrating expression utilizing Arginine/Ornithine transport agents in the Gonads; FIG. 23 is a fluorescent micrograph illustrating expression utilizing Arginine/Ornithine transport agents in the Skin; FIG. 24 is a fluorescent micrograph illustrating expression utilizing Arginine/Ornithine transport agents in the Vessels; and FIG. 25 is a fluorescent micrograph illustrating expression utilizing Arginine/Ornithine transport agents in the Bone Marrow.

The figures illustrate that DNA which codes for the production of green fluorescent protein was distributed throughout all organs and tissues, and successful protein expression has occurred.

1) Example—Delivery of Human Growth Hormone in Mice

Sustained delivery of human growth hormone (hGH) by gene therapy is very challenging. The main reason is that the antigenic nature of hGH elicits a strong antibody response in immunocompetent mice. As a result, hGH delivery reported in the literature is consistently modest (1-3 ng/ml) and transient in nature (lasts for days).

Alginate-PLL-hGH DNA nanoparticles were prepared as described in protocols and mixed with Jell-O. Adult immunocompetent C57BL/6 mice (20 weeks of age) were fed 100 μg of DNA nanoparticles orally (n=3). Mice were bled regularly. The concentration of hGH was determined by ELISA (UBI Inc). The presence of antibodies against hGH was determined by ELISA.

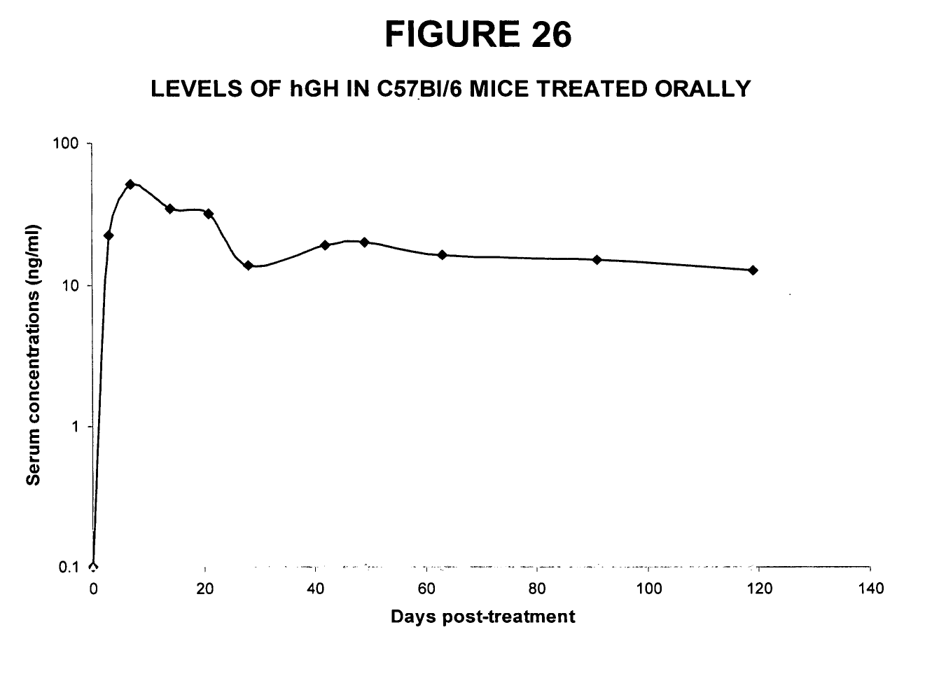

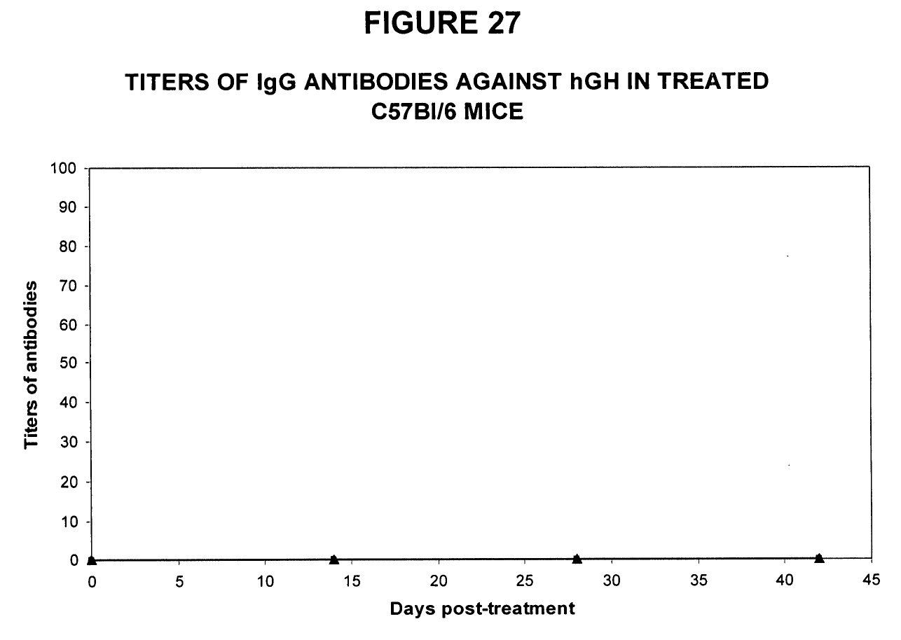

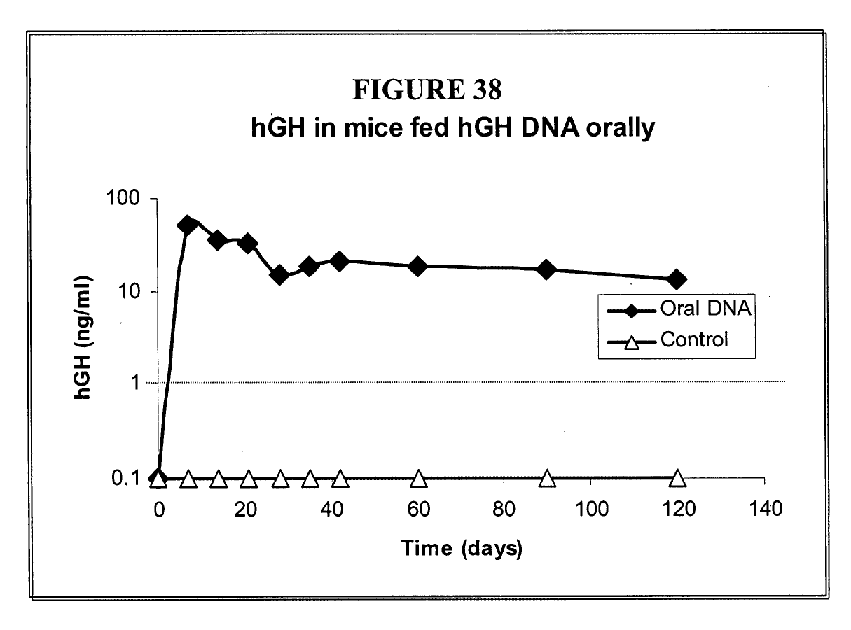

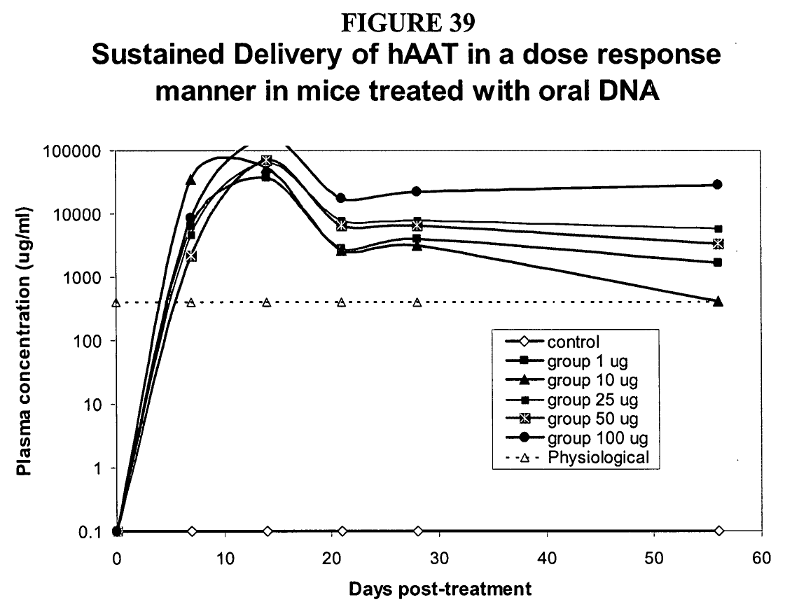

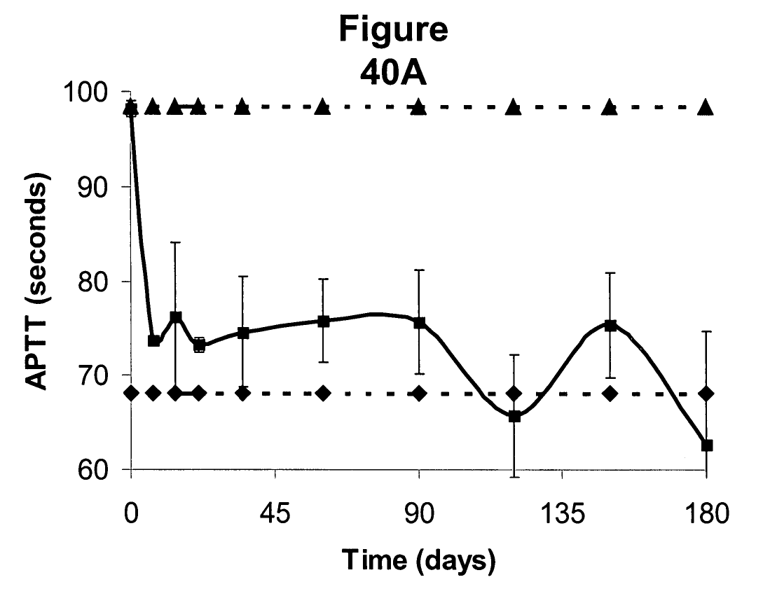

Treated mice had high levels of hGH (peak of 50 ng/ml). More importantly, hGH delivery persisted for at least 120 days (FIGS. 26 and 38). Furthermore, anti-hGH antibodies were not detected (FIG. 27). This data indicates that this technology can deliver sustained levels of therapeutic products such as hGH, without eliciting an antibody response.

Example—Delivery of a Therapeutic Product in a Tissue-Specific Manner in Mice

Tissue specific delivery of hFIX Day 85 post-treatment

A plasmid containing the human factor IX cDNA under the control of the albumin promoter was administered to hemophilic mice, by feeding each mouse 100 micrograms of DNA in alginate-PLL nanoparticle formulation.

The albumin promoter is specific for liver.

hFIX was detected in the blood of treated mice.

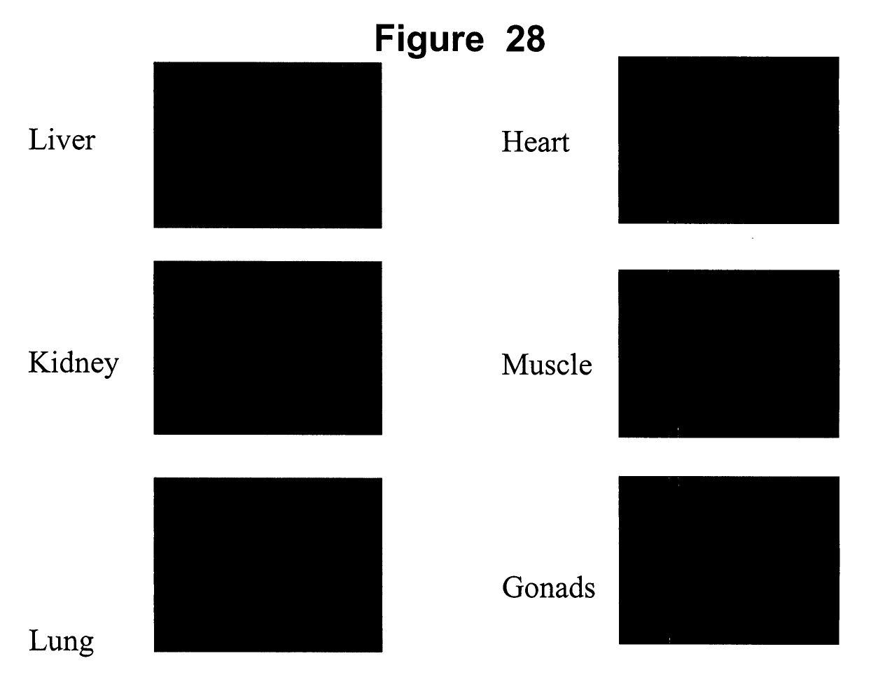

Immunohistochemistry (hFIX present in the various tissues was detected using antibodies specific to hFIX) showed that expression of hFIX in treated mice was restricted to the liver, and was not expressed in other tissues as illustrated in FIG. 28.

This validates the achievement of tissue-specific expression of a transgene following oral administration of DNA.

1) Experimental Protocol:

Alginate-PLL-hFIX DNA nanoparticles were prepared as described in protocols and mixed with Jell-O. The human factor IX (hFIX) DNA was cloned in a plasmid such that the expression of hFIX was placed under the control of the albumin promoter. The albumin promoter is liver-specific. Therefore, expression of hFIX is only expected in liver cells, while cells from other organs harboring this plasmid would not be able to secrete hFIX. Adult immunocompetent C57BL/6 mice (20 weeks of age) were each fed 100 μg of DNA nanoparticles orally (n=3). Mice were bled regularly, and the concentration of hFIX in plasma determined by ELISA (Affinity Biologicals). All treated mice had therapeutic levels of hFIX in blood, while no antibodies were detected.

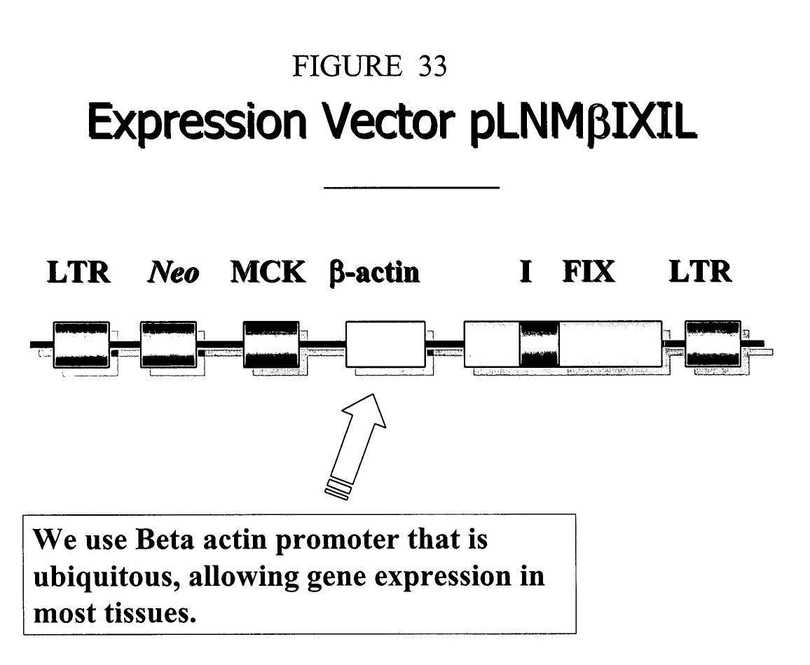

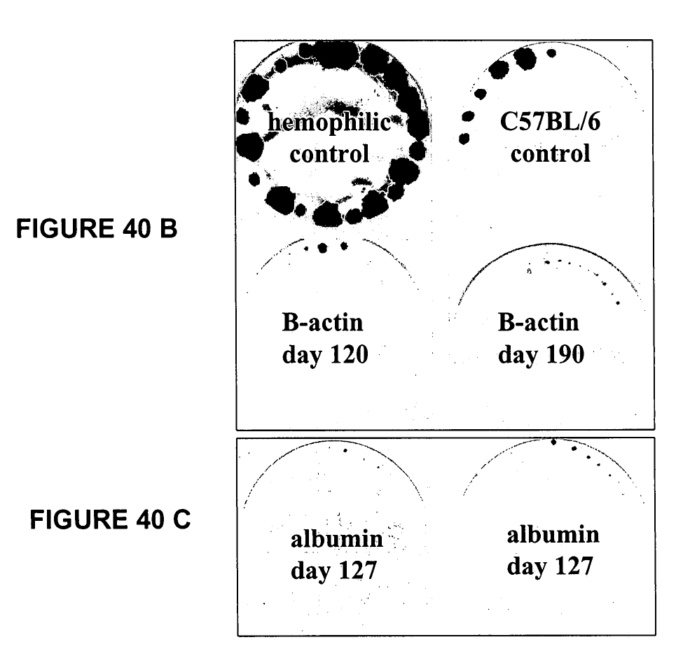

In order to further explore the therapeutic potential of the oral nanoparticles, the following experiment was designed to ascertain whether the oral nanoparticles can “correct” a transgenic mouse suffering from hemophilia B. Factor IX (Christmas factor) is a plasma serine protease necessary for effective blood coagulation. Individuals having absent or defective Factor IX suffer from hemophilia B and are characterized by excessive bleeding. Addition of a functional Factor 1× protein can reverse this genetic defect. Adult factor IX deficient C57BL/6 hemophilia B mice were treated with a single oral dose of 100 ug FIX DNA (pLNMβFIXIL, illustrated in FIG. 33) under the control of the ubiquitous β-actin promoter to ensure expression in all tissues. In order to determine DNA biodistribution, FIX plasmid was amplified and detected in major organs of a mouse sacrificed 120 days post-treatment. Additionally, positive hFIX amplification was detected in organs of a C57BL/6 mouse 400 days after treatment (FIG. 36B). The clotting activity (assessed by the APTT test which measures the efficiency of clotting) of treated hemophilic mice was used to measure transgene expression and the degree of disease correction. By day 3 post-treatment the APTT was corrected in treated mice, and remained within normal range for at least 180 days. In contrast, control mice receiving FIX plasmid alone did not experience any APTT correction. Furthermore, all treated mice had normalized tail bleeding times indicating the phenotypic correction of the disease. Antibodies to hFIX were not detected. By day 89 post-treatment, immunohistochemistry revealed strong hFIX expression in most cells of all major organs. FIG. 40 illustrates the data collected in these experiments to show “correction” (reversal) of hemophilia. FIG. 40A shows the APTT time of treated mice using 1:20 plasma dilution. The APTT of untreated hemophilic mice as well as non-hemophilic C57BL/6 mice are also shown for comparison. Error bars represent the standard deviation. APTT was correction during the duration of the experiment. FIG. 40B shows tail bleeding time. The bleeding time following tail transection of anaesthetized mice was assessed with filter paper every minute and the bleeding used as indication of phenotypic correction. The tail bleeding test was performed only once for each animal. Hemophilic mice treated for 89 days with the plasmid containing the ubiquitous β-actin promoter had bleeding time corrected to normal levels following the oral administration of DNA. FIG. 40C shows that the tail bleeding time of hemophilic mice treated with the plasmid containing the liver-specific albumin promoter was also reduced (127 days). Together all of these results indicate widespread and prolonged hFIX expression both supporting the GFP experimental results and showing the therapeutic potential of the orally administered nanoparticles to reverse hemophilia B.

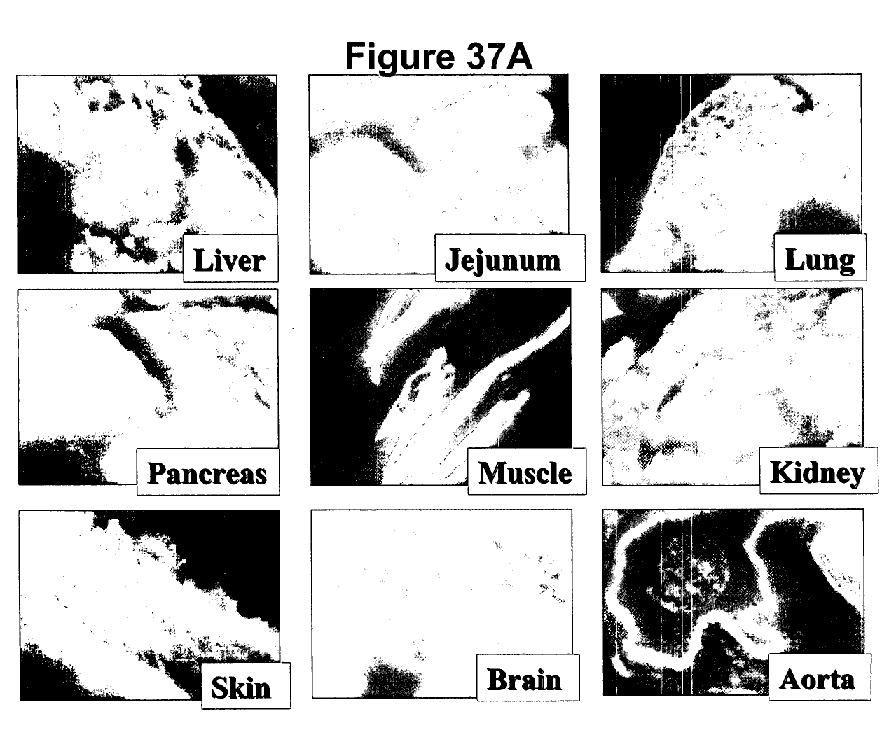

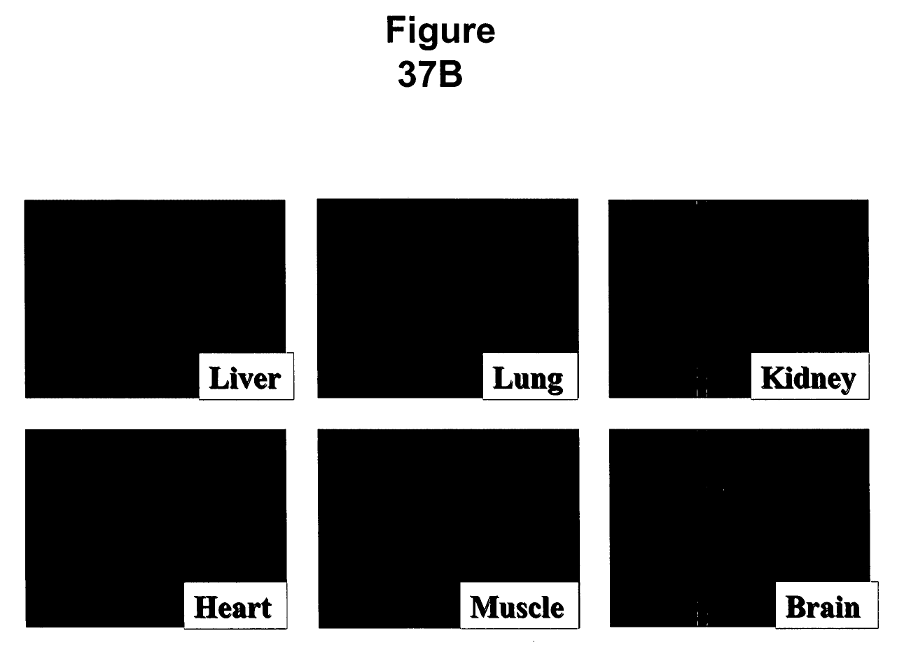

The instant inventors also recognized that widespread gene expression may not be a desirable feature in the treatment of all diseases. The efficient gene expression achieved with hFIX became evident when treated hemophilic mice no longer showed signs of prolonged bleeding but instead developed thrombosis (4/7 mice by day 180). Without being bound by any particular theory, it is suspected that the thrombosis may be caused by supraphysiological production of hFIX in certain tissues. In order to prevent thrombosis and to avoid expression of FIX in all tissues, the experiment described in the immediately proceeding paragraph was repeated using hFIX under the control of a tissue-specific promoter. Hemophilic mice were treated with a plasmid having FIX expression under the control of the liver-specific albumin promoter. The clotting time was shown to be reduced in these treated mice. Tail bleeding was still reduced in mice on day 127. PCR analysis in these mice revealed the presence of the FIX plasmid in all organs, but it was confirmed by immunohistochemistry that FIX expression was restricted to the liver (FIG. 37). In contrast to the mice treated using the ubiquitous β-actin promoter, none of these mice showed any evidence of thrombosis and were all in good health. These findings evidence the potential of this methodology to restrict transgene expression to a desired organ or tissue.

The potential for deleterious germ line transmission is a concern in gene therapy strategies. The instant inventors realized that their method for oral administration of DNA may avoid the potential dangers of germ line transmission. In order to assess germ line transmission through the oral nanoparticles male and female mice were treated with plasmid pLNMβFIXIL containing the cDNA for human factor IX (hFIX) under the control of a ubiquitous β-actin promoter which is active in most tissues. All of the treated animals had circulating levels of hFIX. At this point treated males were bred with similarly treated females. A total of 17 offspring from three different litters were analyzed. In contrast to their parents, none of the offspring had detectable circulating FIX indicating that plasmid pLNMβFIXIL was not active in the offspring. Furthermore, the organs of the offspring had no hFIX DNA as determined by PCR. Human FIX plasmid was not detected in any of the offspring, indicating that the orally administered DNA was not passed on to the offspring. Thus, germline transmission was not evident through the use of the oral nanoparticles.

In order to further strengthen the position that transgenic animals can be generated to produce human therapeutics using the oral nanoparticles the following experiment was carried out. Groups of immunocompetent C57BL/6 mice were administered a single oral dose of alginate nanoparticles containing plasmid palbAAT. This plasmid contains the cDNA for human α-1-antitrypsin under the control of the albumin promoter. Each group of mice received a different concentration of DNA: 1, 10, 25, 50 and 100 ug for each mouse respectively. Mice were bled at regular intervals and the concentration of human α-1-antitrypsin in the circulation of the treated mice was measured by ELISA. By day 15, all treated mice had high levels of therapeutic product, well above the normal physiological concentration (see FIG. 39). Furthermore, the peak of α-1-antitrypsin was dose-dependent and persisted for at least 56 days after treatment (see FIG. 39). Thus as further evidenced by this experiment, the oral nanoparticles can be used to generate transgenic animals to express a human therapeutic.

In summary, the instant inventors have confirmed that orally administered DNA is effectively taken up through the intestine and distributed throughout the body, when protected as it traverses the GI tract by alginate (or any similar agent), and if the DNA is conjugated to a polypeptide (such as PLL). Formulations with no protective coating or no polypeptide evidenced minimal distribution, and very low efficacy and/protein expression. Although not wishing to be limited to any particular theory of operation, it is theorized that DNA is transported to all organs through a natural amino acid distribution mechanism with high efficiency. The DNA enters virtually all cells in all major organs examined and the coded therapeutic product is produced in the various tissues. The inclusion of promoters, either ubiquitous or tissue specific, enable precise control of protein expression.

Delivery is sustained long-term (for at least 180 days). The therapeutic product may be secreted by the cells into the circulation (in the case of secretable products). Alternatively, non-secretable proteins will remain in the cells where they are produced.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification.

One skilled in the art will readily appreciate that the present invention is well adapted to carry out the objects and obtain the ends and advantages mentioned, as well as those inherent therein. The oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. An in vivo process for producing a transgenic animal comprising:
providing at least one transporting agent effective for enabling widespread distribution, systemic expression and sustained delivery of at least one genetic material via a natural gastrointestinal pathway, said genetic material designed to express a biological agent;
forming a distributable moiety by coupling said transporting agent and said genetic material;
administering said distributable moiety to an animal;
transporting said distributable moiety in vivo via said natural gastrointestinal pathway whereby said genetic material is included within essentially all cells of said animal; and
genetically altering said animal via intracellular expression of said biological agent;
wherein a transgenic animal is produced.

2. The process in accordance with claim 1 wherein said animal is a wild-type or a transgenic animal.

3. The process in accordance with claim 1 wherein said step of administering is accomplished rectally.

4. The process in accordance with claim 1 wherein said step of administering is accomplished orally and said process further includes, subsequent to forming said distributable moiety, a step of combining said distributable moiety with a protective agent in a manner effective to protect said distributable moiety during traversal of said natural gastrointestinal pathway.

5. The process in accordance with claim 1 wherein said step of expressing is ubiquitous.

6. The process in accordance with clam 1 wherein said step of expressing is tissue specific.

7. The process in accordance with claim 1 wherein said genetic material comprises at least one complete transcriptional unit.

8. The process in accordance with claim 1 wherein said transporting agent is a polypeptide.

9. The process in accordance with claim 1 wherein said transporting agent is a compound containing an amine group and constructed and arranged to couple with said genetic material to enable widespread distribution, systemic expression and sustained delivery of said genetic material.

10. The process in accordance with claim 1 wherein said coupling is via electrostatic binding.

11. A transgenic animal produced by the process in accordance with claim 1 wherein said animal is characterized by a lack of germ cell alteration thereby precluding progenic transmission.

12. A transgenic animal produced by the process in accordance with claim 2 wherein said animal is characterized by a lack of germ cell alteration thereby precluding progenic transmission.

13. A transgenic animal produced by the process in accordance with claim 3 wherein said animal is characterized by a lack of germ cell alteration thereby precluding progenic transmission.

14. A transgenic animal produced by the process in accordance with claim 4 wherein said animal is characterized by a lack of germ cell alteration thereby precluding progenic transmission.

15. A transgenic animal produced by the process in accordance with claim 5 wherein said animal is characterized by a lack of germ cell alteration thereby precluding progenic transmission.

16. A transgenic animal produced by the process in accordance with claim 6 wherein said animal is characterized by a lack of germ cell alteration thereby precluding progenic transmission.

17. A transgenic animal produced by the process in accordance with claim 7 wherein said animal is characterized by a lack of germ cell alteration thereby precluding progenic transmission.

18. A transgenic animal produced by the process in accordance with claim 8 wherein said animal is characterized by a lack of germ cell alteration thereby precluding progenic transmission.

19. A transgenic animal produced by the process in accordance with. Claim 9 wherein said animal is characterized by a lack of germ cell alteration thereby precluding progenic transmission.

20. A transgenic animal produced by the process in accordance with claim 10 wherein said animal is characterized by a lack of germ cell alteration thereby precluding progenic transmission.
